(19) United States
(12) Patent Application Publication
(10) Pub. No.: US 2006/0172964 A1
(43) Pub. Date: Aug. 3, 2006

(54) Methods for reducing amyloid beta levels

(76) Inventors: Rudolph E. Tanzi, Hull, MA (US); Zhongcong Xie, Andover, MA (US)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 11/344,973

(22) Filed: Feb. 1, 2006

Related U.S. Application Data
(60) Provisional application No. 60/649,039, filed on Feb. 1, 2005.

(51) Int. Cl. A61K 48/00

(52) U.S. Cl. 514/44

(57) ABSTRACT

The present invention relates to the treatment and prophylactic prevention of Alzheimer's disease. More specifically, the present invention relates to methods and compositions for reducing production of β amyloid by reducing or preventing the binding of amyloid precursor protein (APP) to an X11 adaptor protein. Also provided are methods for identifying molecules that modulate APP-X11 binding.

### RELATED APPLICATIONS

This application claims the benefit under 35 U.S.C. §119(e) of U.S. provisional application 60/649,039, filed Feb. 1, 2005, the entire disclosure of which is incorporated herein by reference.

### GOVERNMENT INTEREST

This work was funded in part by the National Institutes of Health under grant numbers AG 014713-07, MH 60009-03, P50 AG05134, and AG 000294-17. The government may have certain rights in this invention.

### FIELD OF THE INVENTION

The present invention relates to the treatment and prophylactic prevention of Alzheimer's disease. More specifically, the present invention relates to methods and compositions for reducing production of β amyloid by reducing or preventing the binding of amyloid precursor protein (APP) to an X11 adaptor protein. Also provided are methods for identifying molecules that modulate APP-X11 binding.

### BACKGROUND OF THE INVENTION

Alzheimer's disease (AD) is one of the greatest public health problems in the U.S., 20 and its impact will only increase in coming decades. AD, an insidious and progressive neurodegenerative disorder accounting for the vast majority of dementia, is characterized by global cognitive decline and the robust accumulation of amyloid deposits and neurofibrillary tangles in the brain.

The study of amyloidogenic APP processing at the gene, protein, and cellular levels has been a major focus of AD neuropathogenesis research since the isolation of APP (amyloid precursor protein) gene in 1987 [see review (1-3)]. Genetic, neuropathological, and biochemical findings indicate that excessive production and/or accumulation of β-amyloid (Aβ) peptide play a fundamental role in the pathogenesis of AD [see review (1-3)]. Aβ is produced from APP through proteolytic processing by two proteases, β-secretase and γ-secretase. Specifically, APP is first hydrolyzed in the extracellular domain, either between Met671 and Asp672, (or between residues 682 and 683), by aspartyl protease β-site APP-cleaving enzyme (BACE) or β-secretase, a type I transmembrane, glycosylated aspartyl protease found in post-Golgi membranes and at the cell surface (4-7). This cleavage by β-secretase generates a 99-residue membrane-associated C-terminus fragment (APP-C99). APP-C99 is further cleaved to release 4-kDa Aβ and β-amyloid precursor protein intracellular domain (AICD). This cleavage is achieved by an unusual form of proteolysis in which the protein is cleaved within the transmembrane domain (at residue +40 or +42) by γ-secretase (8-10). APP, a single-pass and integral transmembrane protein, is more routinely cleaved by α-secretase, at the site close to the transmembrane domain and in the middle of the Aβ region of APP, to release a large ectodomain (α-APPs), leaving a carboxy-terminus fragment of 83 amino acids (APP-C83) in the membrane. While proteolysis of APP-C99 by γ-secretase produces Aβ, proteolysis of APP-C83 by γ-secretase produces p3, a peptide resembling an amino-terminally truncated form of Aβ (11,12), [see review (13)]. Presenilin (PS) and γ-secretase co-fractionate as a detergent-sensitive, high molecular weight complex (14) that includes at least three other proteins, nicastrin/APH2, APH-1, and PEN-2, all of which are necessary for γ-secretase activity [(15-17), see review (18)].

γ-secretase cleavage of the cytoplasmic tail of APP generates the AICD, which contains an absolutely conserved YENPTY (SEQ ID NO:1) motif present in the cytodomains of several tyrosine-kinase receptors (TKR) and in non-receptor tyrosine kinase (TK). In TKR, the tyrosine residue of this motif is phosphorylated upon TK activation and YENPTY-motif (SEQ ID NO:1) functions as a docking site for the phosphotyrosine-binding domain (PTB) present in several adaptor proteins, including X11 family. X11α (MINT 1) and X11β (MINT2), encoded by genes APBA1 and APBA2, respectively, bind to the YENPTY-motif (SEQ ID NO:1) of APP (19,20) [see review (21)].

Over-expression of X11α and X11β has previously been shown to inhibit APP catabolism. Borg et al. (22) and Sastre et al. (23) revealed that over-expression in X11α can increase APP half-life, and can reduce levels of APPs and Aβ in nonneuronal cells (22,23). Like X11α, X11β also stabilizes cellular APP and diminish the levels of APPs and Aβ (24,25). X11α and X11β can interact with presenilin-1 via their PDZ domains (26). Recent studies also showed that over-expression of X11α can impair APP trafficking and may inhibit Aβ production (27). Furthermore, King et al (28) suggested that X11α may specifically interfere with γ-secretase-, but not β-secretase-mediated cleavage of APP and Aβ production.

Effective treatments for AD are lacking while current AD treatments, focusing on downstream events in AD neuropathogenesis, afford only modest, temporary, and palliative benefit. In addition, side effects of treatments are of concern. For example, whereas current γ-secretase inhibitors can decrease Aβ levels, they may also engender intolerable side effects owing to impaired proteolysis of other γ-secretase substrates, such as Notch. Thus, there is a need to decrease Aβ levels, e.g., by specifically and selectively inhibiting γ-secretase cleavage of APP without affecting the processing of other γ-secretase substrates.

### SUMMARY OF THE INVENTION

As a strategy for preventing or treating AD, we propose that interfering with the binding of the APP adaptor proteins, X11α and X11β, to the APP C-terminus will reduce Aβ levels in brains of AD patients while avoiding the potential side effects associated with impairment of the proteolytic processing of other non-APP substrates of γ-secretase, e.g. Notch receptor. This invention includes novel methods for designing and developing therapies aimed at treating and preventing AD by lowering Aβ levels via interfering with the interaction between APP and two of its adapter proteins, X11α and X11β.

Examples of methods to interfere with and reduce APP/X11 binding include: (1) lowering of X11α or X11β levels by RNAi or anti-sense RNA methodologies; (2) lowering of X11α or X11β levels by lowering expression of these molecules at the level of transcription or translation of these molecules; (3) interfering with the binding of APP to X11α or X11β by screening for small molecules capable of such activity; (4) interfering with the binding of APP to X11α or X11β using antibodies to X11α or X11β, or antibodies to the C-terminal portion of APP near the C-terminal YENPTY motif (SEQ ID NO:1) that binds these adaptor molecules; and (5) increasing degradation of X11α and/or X11β polypeptides.

According to one aspect of the invention, methods for reducing the production of Aβ by a cell are provided. The methods include contacting the cell with a molecule that reduces transcription and/or translation of X11α and/or X11β in the cell in an amount effective to reduce the production of Aβ by the cell. In preferred embodiments, the molecule is an APBA1 (encoding X11α) and/or APBA2 (encoding X11β) RNAi molecule. More preferably, the RNAi molecule is a siRNA molecule.

In certain embodiments, the RNAi molecule is formed from two RNA molecules, while in other embodiments the RNAi molecule is a single RNA molecule that forms a double stranded (hairpin) structure.

In still other embodiments, the molecule is an antisense molecule.

According to another aspect of the invention, methods for reducing the production of Aβ by a cell are provided. The methods include contacting the cell with a molecule that reduces binding of APP to X11α and/or APP to X11β in the cell.

The molecule can, in certain embodiments, bind to APP and thereby reduce binding of X11α and/or X11β to APP. In preferred embodiments the molecule is an antibody or an antigen binding fragment thereof that binds to APP, or a polypeptide that comprises a PTB domain, particularly a fragment of X11α protein and/or X11β protein that comprises a PTB domain.

In other embodiments, the molecule binds to X11α and/or X11β and thereby reduces binding of X11α and/or X11β to APP. In preferred embodiments the molecule is an antibody or an antigen binding fragment thereof that binds to X11α and/or X11β, or a polypeptide that comprises a YENPTY sequence (SEQ ID NO:1). In preferred embodiments, the polypeptide is a fragment of APP.

In some embodiments, the contacting occurs in vitro.

According to still another aspect of the invention, methods for treating or preventing Alzheimer's disease in a subject are provided. The methods include reducing transcription and/or translation of X11α and/or X11β nucleic acids. In certain embodiments, the transcription and/or translation of X11α protein and/or X11β protein is reduced by administering to the subject one or more molecules that bind to X11α and/or X11β nucleic acids and block transcription and/or translation of the X11α and/or X11β nucleic acids, in an amount effective to reduce the transcription and/or translation of X11α and/or X11β, nucleic acids and effective to reduce the production of amyloid β.

In some embodiments, the one or more molecules that bind to X11α and/or X11β nucleic acids and block transcription and/or translation are APBA1 (encoding X11α) and/or APBA2 (encoding X11β) RNAi molecules. The RNAi molecules preferably are siRNA molecules, which can be formed from two RNA molecules or a single RNA molecule that forms a double stranded (hairpin) structure. Alternatively, the one or more molecules that bind to X11α and/or X11β nucleic acids and block transcription and/or translation are antisense nucleic acid molecules.

According to yet another aspect of the invention, methods for treating or preventing Alzheimer's disease in a subject are provided. The methods include reducing binding of amyloid precursor protein (APP) to X11α protein and/or X11β protein by administering to the subject one or more molecules that bind to APP and block binding of X11α and/or X11β to a YENPTY sequence (SEQ ID NO:1) of APP and/or one or more molecules that bind to X11α and/or X11β and block binding of APP to a phosphotyrosine-binding domain (PTB) of X11α and/or X11β, in an amount effective to reduce the binding of X11α and/or X11β to APP and to reduce the production of amyloid β.

In preferred embodiments the molecule is an antibody or an antigen binding fragment thereof that binds to APP protein, preferably to a YENPTY sequence (SEQ ID NO:1).

The molecule in other embodiments is a polypeptide that comprises a YENPTY sequence (SEQ ID NO:1), preferably a fragment of APP that comprises a YENPTY sequence (SEQ ID NO:1).

In other embodiments, the molecule is an antibody or an antigen binding fragment thereof that binds to X11α protein and/or X11β protein. Preferably the antibody or the antigen binding fragment thereof binds to a PTB domain of X11α protein and/or X11β protein.

In further embodiments, the molecule is a polypeptide that comprises a PTB domain, preferably a fragment of X11α protein and/or X11β protein that comprises a PTB domain.

The animal preferably is a human.

Methods for identifying molecules that reduce production of amyloid β (Aβ) by reducing the binding of amyloid precursor protein (APP) to X11α protein and/or X11β protein are provided according to a further aspect of the invention. The methods include providing a reaction mixture that includes APP and/or a fragment thereof that includes a YENPTY sequence (SEQ ID NO:1), and X11α, X11β and/or a fragment thereof that binds to APP. The methods also include contacting the reaction mixture with a candidate inhibitor molecule, determining a level of binding of the APP or the fragment thereof with X11α, X11β and/or the fragment thereof in the absence and in the presence of the candidate inhibitor molecule, and comparing the level of binding of APP or the fragment thereof with X11α, X11β and/or the fragment thereof in the absence and in the presence of the candidate inhibitor molecule. A reduction in the binding in the presence of the candidate inhibitor molecule relative to the level of binding in the absence of the candidate inhibitor molecule indicates that the candidate inhibitor molecule is a molecule that reduces production of Aβ.

In preferred embodiments, the candidate inhibitor molecule is a small molecule; an antibody that binds to APP or X11α protein and/or X11β protein, or an antigen-binding fragment thereof; a polypeptide that comprises a YENPTY sequence (SEQ ID NO:1), preferably a fragment of APP; or a polypeptide that comprises a phosphotyrosine binding (PTB) domain, preferably a fragment of X11α protein or X11β protein.

In some embodiments, the reaction mixture is a cell.

Also provided according to another aspect of the invention are methods for identifying molecules that reduce production of amyloid β (Aβ) by reducing transcription and/or translation of X11α and/or X11β. The methods include providing a reaction mixture comprising X11α and/or X11β nucleic acids and transcription and/or translation machinery sufficient to transcribe and/or translate X11α and/or X11β nucleic acids, contacting the reaction mixture with a candidate inhibitor molecule under conditions that permit transcription and/or translation of the X11α and/or X11β nucleic acids, determining the transcription and/or translation of the X11α and/or X11β nucleic acids, and comparing the level of transcription and/or translation of the X11α and/or X11β nucleic acids in the absence and in the presence of the candidate inhibitor molecule. A reduction in the level of transcription and/or translation in the presence of the candidate inhibitor molecule relative to the level of level of transcription and/or translation in the absence of the candidate inhibitor molecule indicates that the candidate inhibitor molecule is a molecule that reduces production of Aβ.

In preferred embodiments, the candidate inhibitor molecule is a small molecule, or a siRNA molecule. Preferably the reaction mixture is a cell.

According to another aspect of the invention, methods for identifying molecules that reduce production of amyloid β (Aβ) by increasing degradation of X11 molecules are provided. The methods include providing a cell that expresses X11 molecules, contacting the cell with a candidate degradation enhancer molecule under conditions that permit degradation of the X11 molecules, determining the degradation of the X11 molecules, and comparing the level of degradation of the X11 molecules in the absence and in the presence of the candidate degradation enhancer molecule, wherein an increase of degradation in the presence of the candidate degradation enhancer molecule relative to the level of degradation in the absence of the candidate degradation enhancer molecule indicates that the candidate degradation enhancer molecule is a molecule that reduces production of Aβ.

In preferred embodiments, the candidate degradation enhancer molecule is a small molecule; a siRNA molecule; a nucleic acid molecule that encodes a polypeptide that increases degradation, preferably including an expression vector; or a polypeptide that increases degradation.

The X11 molecules in various embodiments are X11α polypeptides, X11β polypeptides, X11α nucleic acids and/or X11β nucleic acids.

In another aspect, the invention provides for use of the foregoing agents, compounds and molecules in the preparation of medicaments, particularly medicaments for the treatment of Alzheimer's disease and other conditions in which reduced production of Aβ is therapeutically or prophylactically beneficial.

These and other aspects of the invention will be described in greater detail below.

### BRIEF DESCRIPTION OF THE DRAWINGS

FIG. 1 shows the effects of APBA1 RNAi on APP processing and Aβ levels in H4-APP-FL cells. In H4-APP-FL cells, APBA1 small interfering RNA (siRNA) treatment decreased the protein levels of X11α, increased the protein levels of APP-C83 and APP-C99, and decreased Aβ production. FIG. 1A: APP processing in Western blot analyses. X11α immunoblotting showed reductions in the protein levels of X11α: in the cells treated with APBA1 siRNA (columns 4 to 6) as compared to control siRNA (columns 1 to 3) or saline (7 to 9). APP-FL immunoblotting revealed that there was no significant difference in the protein levels of APP-FL in the cells treated with control siRNA, APBA1 siRNA, or DAPT. Synthetic forms of APP-C99 and APP-C83 were used as markers to confirm the bands of APP-C99 and APP-C83 in the blot. APP-CTF immunoblotting showed increases in the protein levels of APP-C99 and APP-C83 in the cells treated with APBA1 siRNA (columns 4 to 6) or DAPT (columns 7 to 9) as compared to control siRNA (columns 1 to 3). The blot showing the band of APP-C83 only was the same blot with less exposure time in developing the film. The protein levels of APP-FL, APP-C83 and APP-C99 in H4 cells over-expressing APP-FL (columns 1 to 9) are higher than those in H4 naïve cells (columns 10 and 11). There was no significant difference in the amounts of β-actin in the control siRNA-, APBA1 siRNA- and DAPT-treated H4-APP-FL cells, and H4 naïve cells. FIG. 1B: The protein levels of X11α assessed by quantifying X11α: in the Western blot. APBA1 siRNA treatment (black bar) significantly decreased the protein levels of X11α as compared to control siRNA treatment (white bar) (*p<0.05), normalized to β-actin. FIG. 1C: APP processing assessed by quantifying the ratio of APP-C99 to APP-FL in the Western blot. APBA1 siRNA treatment (black bar) or DAPT treatment (gray bar) significantly increased the ratio of APP-C99 to APP-FL as compared to control siRNA treatment (white bar) (*p<0.05), normalized to β-actin. FIG. 1D: APP processing assessed by quantifying the ratio of APP-C83 to APP-FL in the Western blot. APBA1 siRNA treatment (black bar) or DAPT treatment (gray bar) significantly increased the ratio of APP-C83 to APP-FL as compared to control siRNA treatment (white bar) (*p<0.05), normalized to β-actin. FIG. 1E: Effects of APBA1 RNAi on Aβ production in H4-APP-FL cells. APBA1 siRNA treatment (black bar) or DAPT treatment (gray bar) decreased Aβ₄₀ production as compared to control siRNA treatment (white bar) (*p<0.05).

FIG. 2 shows the effects of APBA2 RNAi on APP processing and Aβ levels in H4-APP-FL cells. In H4-APP-FL cells, APBA2 siRNA treatment decreased the protein levels of X11β, but did not alter the protein levels of APP-FL, APP-C83 and APP-C99. APBA2 siRNA treatment decreased Aβ production. FIG. 2A: APP processing in Western blot analyses. X11β immunoblotting showed reductions in the protein levels of X11β in the cells treated with APBA2 siRNA (columns 4 to 6) as compared to control siRNA (columns 1 to 3) or saline (7 to 9). APP-FL immunoblotting revealed that there was no significant difference in the protein levels of APP-FL in the cells treated with control siRNA, APBA2 siRNA or DAPT. Synthetic forms of APP-C99 and APP-C83 were used as markers to confirm the bands of APP-C99 and APP-C83 in the blot. APP-CTF immunoblotting showed increases in the protein levels of APP-C99 and APP-C83 in the cells treated with DAPT (columns 7 to 9), but not APBA2 siRNA (columns 4 to 6), as compared to controls (columns 1 to 3). The protein levels of APP-FL, APP-C83 and APP-C99 in the H4-APP-FL cells (columns 1 to 9) are higher than those in H4 naïve cells (columns 10 and 11). β-Actin immunoblotting revealed that there was no significant difference in the amounts of β-actin in the control siRNA-, APBA2 siRNA- or DAPT-treated H4-APP-FL cells, and H4 naïve cells. FIG. 2B: The protein levels of X11β assessed by quantifying X11β in the Western blot. APBA2 siRNA treatment (black bar) significantly decreased the protein levels of X11β as compared to control siRNA treatment (white bar) (*p<0.05), normalized to β-actin. FIG. 2C: APP processing assessed by quantifying the ratio of APP-C99 to APP-FL in the Western blot. APBA2 siRNA treatment (black bar) did not alter the ratio of APP-C99 to APP-FL as compared to control siRNA treatment (white bar), normalized to β-actin. DAPT treatment (gray bar) increased the ratio of APP-C99 to APP-FL as compared to control treatment (*p<0.05), normalized to β-actin. FIG. 2D: APP processing assessed by quantifying the ratio of APP-C83 to APP-FL in the Western blot. APBA2 siRNA treatment (black bar) did not alter the ratio of APP-C83 to APP-FL as compared to control siRNA treatment (white bar), normalized to β-actin. DAPT treatment (gray bar) increased the ratio of APP-C83 to APP-FL as compared to control treatment (*p<0.05), normalized to β-actin. FIG. 2E: Effects of APBA2 RNAi on Aβ production in H4-APP-FL cells. Both APBA2 siRNA (black bar) and DAPT (gray bar) treatments decreased Aβ₄₀ production as compared to control siRNA treatment (white bar) (*p<0.05).

FIG. 3 shows the effects of APBA1 RNAi on APP processing and Aβ levels in H4-APP-C99 cells. In H4-APP-C99 cells, APBA1 siRNA treatment decreased the protein levels of X11α, increased the protein levels of APP-C83 and APP-C99 and decreased Aβ production. FIG. 3A: APP processing in Western blot analyses. X11α immunoblotting showed reductions in the protein levels of X11α in the cells treated with APBA1 siRNA (columns 5 to 7) as compared to control siRNA (columns 2 to 4). APP-FL immunoblotting revealed that there was no significant difference in the protein levels of APP-FL in the cells treated with control siRNA or APBA1 siRNA. Synthetic forms of APP-C99 and APP-C83 were used as markers (column 1) to confirm the bands of APP-C99 and APP-C83 in the blot. APP-CTF immunoblotting showed increases in the protein levels of APP-C99 and APP-C83 in the cells treated with APBA1 siRNA (columns 5 to 7) as compared to control siRNA (columns 2 to 4). The blot showing the band of APP-C83 only was the same blot with less exposure time in developing the film. The protein levels of APP-C83 and APP-C99 in the H4-APP-C99 cells (columns 2 to 7) are higher than those in the H4 naïve cells (columns 8 and 9). There was no significant difference in the amounts of β-actin in the control siRNA- or APBA1 siRNA-treated H4-APP-C99 cells, and H4 naïve cells. FIG. 3B: X11α protein levels assessed by quantifying X11α in the Western blot. APBA1 siRNA treatment significantly decreased the protein levels of X11α as compared to control siRNA treatment (*p<0.05), normalized to β-actin. FIG. 3C: APP processing assessed by quantifying the ratio of APP-C99 to APP-FL in the Western blot. APBA1 siRNA treatment significantly increased the ratio of APP-C99 to APP-FL as compared to control siRNA treatment (*p<0.05), normalized to β-actin. FIG. 3D: APP processing assessed by quantifying the ratio of APP-C83 to APP-FL in the Western blot. APBA1 siRNA treatment significantly increased the ratio of APP-C83 to APP-FL as compared to control siRNA treatment (*p<0.05), normalized to β-actin. FIG. 3E: Effects of APBA1 RNAi on Aβ production in H4-APP-C99 cells. APBA1 siRNA treatment decreased Aβ₄₀ production as compared to control siRNA treatment (*p<0.05).

FIG. 4 shows the effects of APBA2 RNAi on APP processing and Aβ levels in H4-APP-C99 cells. In H4-APP-C99 cells, APBA2 siRNA treatment decreased the protein levels of X11β, but did not alter the protein levels of APP-FL, APP-C83 and APP-C99. APBA2 siRNA treatment did not alter Aβ production. FIG. 4A: APP processing in Western blot analyses. X11β immunoblotting showed reductions in the protein levels of X11β in the cells treated with APBA2 siRNA (columns 6 to 8) as compared to control siRNA (columns 3 to 5). APP-FL immunoblotting revealed that there was no significant difference in the protein levels of APP-FL in the cells treated with control siRNA or APBA2 siRNA. Synthetic forms of APP-C99 and APP-C83 were used as markers (column 1) to confirm the bands of APP-C99 and APP-C83 in the blot. APP-CTF immunoblotting showed no significant differences in the protein levels of APP-C99 and APP-C83 in the cells treated with APBA2 siRNA (columns 6 to 8) as compared to control siRNA (columns 3 to 5). The blot showing the bands of APP-C83 only was the same blot with less exposure time in developing the film. The protein levels of APP-C83 and APP-C99 in the H4-APP-C99 cells (columns 3 to 8) are higher than those in H4 naïve cells (columns 2 and 9). There was no significant difference in the amounts of β-actin in the control siRNA- or APBA2 siRNA-treated H4-APP-C99 cells, and H4 naïve cells. FIG. 4B: X11β protein levels assessed by quantifying X11β in the Western blot. APBA2 siRNA treatment significantly decreased the protein levels of X11β as compared to control siRNA treatment (*p<0.05), normalized to β-actin. FIG. 4C: APP processing assessed by quantifying the ratio of APP-C99 to APP-FL in the Western blot. APBA2 siRNA treatment did not alter the ratio of APP-C99 to APP-FL as compared to control siRNA treatment, normalized to β-actin. FIG. 4D: APP processing assessed by quantifying the ratio of APP-C83 to APP-FL in the Western blot. APBA2 siRNA treatment did not alter the ratio of APP-C83 to APP-FL as compared to control siRNA treatment, normalized to β-actin. FIG. 4E: Effects of APBA2 RNAi on Aβ production in H4-APP-C99 cells. APBA2 siRNA treatment did not change Aβ₄₀ production as compared to control siRNA treatment.

### DETAILED DESCRIPTION OF THE INVENTION

To date, the effects of reduced expression of APBA2 and APBA2, or X11α and X11β, on APP processing and Aβ production have not been assessed. To examine the effects of reduced expression of X11α and X11β on APP processing and Aβ production, we established RNA interference (RNAi) for APBA1 and APBA2 in H4 cells over-expressing either APP-FL or APP-C99, and evaluated the effects of RNAi-mediated silencing of APBA1 and APBA2 on APP processing and Aβ production. Surprisingly, we determined that RNAi silencing of APBA1 in H4 human neuroglioma cells stably transfected to express either APP-FL or APP-C99 increased levels of APP-C-terminal fragments (APP-CTFs) and lowered Aβ levels in both cell lines by inhibiting γ-secretase cleavage of APP. Meanwhile, RNAi silencing of APBA2 also lowered Aβ levels but apparently not via attenuation of γ-secretase cleavage of APP. The notion of attenuating γ-secretase cleavage of APP via the APP adaptor protein, X11α, is particularly attractive with regard to therapeutic potential, given that side effects of γ-secretase inhibition owing to impaired proteolysis of other γ-secretase substrates, e.g. Notch, might be avoided.

Thus, we propose that interfering with the binding of the APP adaptor proteins, X11α and X11β, to the APP C-terminus (also referred to herein as “APP/X11 binding”) will reduce Aβ levels in brains of AD patients, which is useful for preventing or treating AD.

Examples of methods to interfere with and reduce APP/X11 binding include: (1) lowering of X11α or X11β levels by RNAi or anti-sense RNA methodologies; (2) lowering of X11α or X11β levels by lowering expression of these molecules at the level of transcription or translation of these molecules; (3) interfering with the binding of APP to X11α or X11β by screening for small molecules capable of such activity; (4) interfering with the binding of APP to X11α or X11β using antibodies to X11α or X11β, or antibodies to the C-terminal portion of APP near the C-terminal YENPTY motif (SEQ ID NO:1) that binds these adaptor molecules; and (5) increasing degradation of X11α and/or X11β polypeptides.

As used herein, “X11” means X11α and/or X11β, and “X11 molecules” means X11 nucleic acids and/or polypeptides unless the context is limiting.

A reduction in expression of a X11 nucleic acid or polypeptide may be achieved, in certain preferred embodiments, by using the technique of RNA interference (RNAi). The use of RNAi involves the use of double-stranded RNA (dsRNA) to block or reduce gene expression. (See, e.g.: Sui, G, et al, 2002, Proc Natl. Acad. Sci. U.S.A. 99:5515-5520). The application of RNAi methodologies for specifically reducing gene expression is understood by one of ordinary skill in the art. By “specifically reducing gene expression” is meant that the practitioner can reduce or even eliminate expression of a specific gene or genes, without substantially altering expression of other genes.

As proof of principle, we have used small interfering RNAs (siRNAs) to reduce expression of the APP-binding adaptor proteins X11α and X11β, and analyzed the effects on APP proteolysis and Aβ production.

Reduction of the binding of X11 polypeptides to APP can be accomplished by a variety of methods in addition to reducing expression of X11 polypeptides, including by use of antibodies that bind to the X11 molecules or to APP, peptides (or peptide mimetics) that bind to X11 molecules at the APP binding site and peptides (or peptide mimetics) that bind to APP molecules at the X11 binding site. In addition, other types of molecules (e.g., small organic molecules) that reduce transcription and/or translation or X11 molecules, or reduce binding of X11 polypeptides to APP also can be used in accordance with the invention. These are described in greater detail elsewhere herein.

In one aspect of the invention, a method is provided in which siRNA molecules are used to reduce the expression of X11 molecules. In one embodiment, a cell is contacted with a small interfering RNA (siRNA) molecule to produce RNA interference (RNAi) that reduces expression of one or more X11 molecules (e.g., X11α and/or X11β). The siRNA molecule is directed against nucleic acids coding for the X11 molecule (e.g. RNA transcripts including untranslated and translated regions). Exemplary X11α and X11β siRNA molecules, which are not limiting of the siRNA molecules that can be used in accordance with the invention, are provided in the Examples The expression level of the targeted X11 molecule(s) can be determined using well known methods such as Western blotting for determining the level of protein expression and Northern blotting or RT-PCR for determining the level of mRNA transcript of the target gene, some of which are shown in the Examples below.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of a X11 nucleic acid molecule is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of X11α or X11β. Combinations of the foregoing can be expressed from a single vector or from multiple vectors introduced into cells.

In one aspect use of the invention a vector comprising any of the nucleotide sequences of the invention is provided, preferably one that includes promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2NV5-GW/lacZ, pENTR/U6, pLenti6-GW/U6-laminshrna and pLenti6/BLOCK-iT-DEST (Invitrogen). These vectors and others are available from commercial suppliers.

According to an aspect of the invention, a vector comprising any of the isolated nucleic acid molecules of the invention, operably linked to a promoter to produce siRNA molecules is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. An expression vector is one into which a desired DNA sequence may be inserted by restriction and ligation such that it is operably joined to regulatory sequences and may be expressed as an RNA transcript.

Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein.

As used herein, a coding sequence and regulatory sequences are said to be “operably joined” or “operably linked” when they are covalently linked in such a way as to place the expression or transcription of the coding sequence under the influence or control of the regulatory sequences. As used herein, “operably joined” and “operably linked” are used interchangeably and should be construed to have the same meaning.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the X11 nucleic acid molecules in expression vectors (for example to produce siRNA), as well as to transfect host cells and cell lines, for example eukaryotic cells. Especially useful are mammalian cells such as human, mouse, hamster, pig, goat, primate, etc. In one aspect of the invention the cells may be neuronal cells.

The invention, in one aspect, also permits the construction of X11 gene “knock-outs” or “knock-downs” in cells and in animals, providing materials for studying certain aspects of APP processing, Aβ production, and Alzheimer's disease. For example, a knock-out mouse (gene disruption) or a knock-down mouse (reduced gene expression by e.g., siRNA) may be constructed and examined for clinical parallels between the model and a mouse having Alzheimer's disease-like symptoms and/or physiology that is treated to downregulate expression of a X11 molecule.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection using Effectene (Qiagen) or other commercially available transfection aids, transfection or infection with viruses including the nucleic acid of interest, liposome mediated transfection, electroporation, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies that selectively bind a cell surface antigen, particularly those that are readily internalized.

In one aspect of the invention a method is provided for targeting a nucleic acid molecule to a cell for therapeutic use. In a preferred embodiment of the invention the nucleic acid molecule is delivered intracellularly. In a further embodiment of the invention, an antibody is used to target a nucleic acid molecule to a cell. In yet another embodiment of the invention, an antibody can be delivered alone or together with a nucleic acid molecule. In a further aspect, an antibody is delivered in combination with a delivery vehicle, such as a liposome. The antibody includes whole antibody or fragments of antibody as is described in greater detail below.

Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Antibodies that bind X11 molecules or APP can be used to reduce the binding of X11 molecules to APP. Preferred antibodies include antibodies that bind to the YENPTY (SEQ ID NO:1) sequence of APP or the phosphotyrosine-binding domain (PTB) present in X11α and X11β. Other antibodies that bind to other epitopes on APP or X11 polypeptides and block or reduce binding of APP to X11 (through steric hindrance, conformational change or other mechanisms) also can be used. To determine inhibition, a variety of assays known to one of ordinary skill in the art can be employed. For example, the protein binding assays can be used to determine if an antibody inhibits the binding of X11 molecules to APP.

As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The invention also includes the use of antigen-binding fragments of antibodies in the same manner as antibodies are used in any of the methods disclosed herein. The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

An “isolated antibody”, as used herein, is intended to refer to an antibody which is substantially free of other antibodies having different antigenic specificities (e.g., an isolated antibody that specifically binds to APP or an X11 polypeptide, such as X11α or X11β, is substantially free of antibodies that specifically bind antigens other than these molecules). An isolated antibody that specifically binds to an epitope, isoform or variant of APP or an X11 polypeptide may, however, have cross-reactivity to other related antigens, e.g., from other species (i.e., APP or X11 orthologs). Moreover, an isolated antibody may be substantially free of other cellular material and/or chemicals, although it need not be. As used herein, “specific binding” refers to antibody binding to a predetermined antigen. Typically, the antibody binds with an affinity that is at least two-fold greater than its affinity for binding to a non-specific antigen (e.g., BSA, casein) other than the predetermined antigen or a closely-related antigen.

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment. Alternatively, the fragments are “domain antibody fragments”. Domain antibodies are the smallest binding part of an antibody (approximately 13 kDa). Examples of this technology are disclosed in U.S. Pat. Nos. 6,248,516, 6,291,158, 6,127,197 and EP patent 0368684.

As used herein, antibodies also include single chain antibodies (e.g., scFvs). In some embodiments, the single chain antibodies are disulfide-free antibodies having mutations, e.g., in disulphide bond forming cysteine residues. The antibodies may be prepared by starting with any of a variety of methods, including administering protein, fragments of protein, cells expressing the protein or fragments thereof and the like to an animal to induce polyclonal antibodies, Such antibodies or antigen-binding fragments thereof may be used in the preparation of scFvs and disulfide-free variants thereof. The antibodies or antigen-binding fragments thereof may be used, for example, to reduce binding of APP and X11 molecules, and to identify molecules that bind APP or X11 molecules (as determined by competition with the antibody for binding to APP or X11 molecules).

Various forms of the antibody polypeptide or encoding nucleic acid can be administered and delivered to a mammalian cell (e.g., by virus or liposomes, or by any other suitable methods known in the art or later developed). The method of delivery can be modified to target certain cells, and in particular, cell surface receptor molecules or antigens present on specific cell types. Methods of targeting cells to deliver nucleic acid constructs, for intracellular expression of the antibodies (i.e., as “intrabodies”), are known in the art. In these applications, single chain antibodies are generally used, and the size of the antibody (or fragment) is kept to a minimum to facilitate translocation into the cell. The antibody polypeptide sequence can also be delivered into cells by providing a recombinant protein fused with peptide carrier molecules. These carrier molecules, which are also referred to herein as protein transduction domains (PTDs), and methods for their use, are known in the art. Examples of PTDs, though not intended to be limiting, are tat, antennapedia, and synthetic poly-arginine; nuclear localization domains also can be included in the antibody molecules. These delivery methods are known to those of skill in the art and are described in U.S. Pat. No. 6,080,724, and U.S. Pat. No. 5,783,662, the entire contents of which are hereby incorporated by reference.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known and are disclosed for example in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with APP, X11α or X11β. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. See; Goding (in Monoclonal Antibodies: Principles and Practice, 2d ed., pp. 60-61, Orlando, Fla., Academic Press, 1986).

The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elsevier, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which that relate to preparation of human antibodies are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. In particular, mouse strains that have human immunoglobulin genes inserted in the genome (and which cannot produce mouse immunoglobulins) are preferred. Examples include the HuMAb mouse strains produced by Medarex/GenPharm International, and the XenoMouse strains produced by Abgenix. Such mice produce fully human immunoglobulin molecules in response to immunization.

The invention also embraces fusion proteins comprising all or part of the X11α and X11β amino acid sequences. The preparation and use of fusion proteins is a well known method to those of skill in the art. Examples of fusion proteins include but are not limited to GST, green fluorescent protein (GFP), histidine tags, and red fluorescent protein.

As noted above, reduction in binding of X11 polypeptide to APP also can be accomplished using peptides or peptide mimetics that bind to X11 molecules at the APP binding site and peptides or peptide mimetics that bind to APP molecules at the X11 binding site. More generally, the peptides or peptide mimetics need not bind to the exact binding site of X11 polypeptides for APP or of APP for X11 polypeptides, but can bind to a site distal to the binding site and reduce binding of the APP and X11 polypeptides to each other. Examples of this are peptides or peptide mimetics that bind and sterically hinder binding of X11 polypeptides and APP, and peptides or peptide mimetics that bind and induce a conformational change in the bound protein such that binding of X11 polypeptides and APP is reduced.

Peptides useful in accordance with the invention include peptides that include a YENPTY sequence (SEQ ID NO:1), which is present in APP and is required for X11 polypeptide binding to APP. The peptides can be, or include, fragments of APP that include the YENPTY sequence (SEQ ID NO:1). Other useful peptides are peptides that include a phosphotyrosine binding domain (PTB), preferably those of the X11α or X11β polypeptides. The peptides can be, or include, fragments of X11α or X11β.

“Peptide mimetics”, as used herein, are non-peptide molecules that mimic the structure of peptides. Peptide mimetics represent one class of small molecules that can be screened for using the screening assays for identifying pharmacological agents of the invention as described herein.

As used herein with respect to polypeptides, proteins or fragments thereof and peptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

The invention also pertains to decreasing APP/X11 binding, and thereby decreasing Aβ production, by increasing degradation of X11α and/or X11β molecules, particularly X11 polypeptides. It is anticipated that certain antibodies, antibody fragments, and other molecules that bind X11 molecules will increase degradation of X11 molecules. The screening methods of the invention, presented in greater detail below, can be used to identify such molecules that bind to X11 molecules as increase degradation thereof.

The invention further provides efficient methods for identifying pharmacological agents or lead compounds for agents useful for decreasing the binding of APP and X11α or X11β, for decreasing the transcription and/or translation of X11 molecules, or for increasing degradation of X11 molecules, and thereby decreasing production of Aβ. Such methods are adaptable to automated, high throughput screening of compounds.

A wide variety of assays for pharmacological agents that reduce production of Aβ are provided, including assays for determining affinity and binding of APP and X11 proteins, e.g., immunoprecipitations, two-hybrid assays, binding assays etc., and assays for determining the effect on transcription and/or translation of gene sequences (e.g., APBA1, APBA2) or degradation of X11 molecules. For example, protein-protein binding assays using APP and X11α can be used to determine directly if a candidate pharmacological agent reduces binding of these proteins, and two-hybrid assays can be used to determine indirectly if a candidate pharmacological agent reduces binding of these proteins. Various assays for determining transcription and/or translation of a nucleic acid molecule can be used to determine the effects of a candidate pharmacological agent on the APBA1 and APBA2 gene sequences (recombinant or genomic). Degradation of X11α and/or X11β molecules can be determined using, for example, standard assays of polypeptides to measure the rate and/or amount of X11 polypeptide degradation, or standard assays of nucleic acids to measure the rate and/or amount of X11 nucleic acid degradation or decrease in stability. The candidate pharmacological agents are also referred to herein as candidate inhibitor molecules or candidate degradation enhancer molecules.

The candidate pharmacological agents can be derived from, for example, combinatorial peptide libraries, small molecule libraries, natural product libraries or expression libraries of gene sequences.

A typical assay for measuring protein binding includes forming a reaction mixture of two proteins being assayed for binding (e.g., APP and X11α), or fragments of the proteins that contain the respective protein binding sites, which mixture is contacted with a candidate inhibitor molecule before or after binding of the proteins. The level of binding of the proteins in the absence or presence of the candidate inhibitor molecule then is determined, using standard procedures as described in the Examples and as are well known in the art, which can be compared to a control level of binding. For example, binding can be determined using immunological assays (e.g., pull-down assays), yeast two hybrid assays, chromatographic methods (HPLC, FPLC, optionally coupled to mass spectrometry), fluorescence resonance energy transfer assays, surface plasmon resonance assays, etc. A reduction in the binding of the proteins in the presence of the candidate inhibitor molecule relative to the level of binding in the absence of the candidate inhibitor molecule indicates that the candidate inhibitor molecule is a molecule that reduces production of Aβ. The reaction mixture can be a cell (in vitro or in vivo) or a mixture of isolated and/or purified components.

A typical assay for measuring transcription and/or translation includes forming a reaction mixture of X11α and/or X11β nucleic acids and transcription and/or translation machinery sufficient to transcribe and/or translate the X11α and/or X11β nucleic acids. The reaction mixture is contacted with a candidate inhibitor molecule under conditions that permit transcription and/or translation of the X11α and/or X11β nucleic acids. The amount of transcription and/or translation of the X11α and/or X11β nucleic acids then is determined using standard procedures as described in the Examples and as are well known in the art. For example, transcription can be determined by assays of nucleic acids including PCR assays, run-off transcription assays, hybridization assays (e.g., Northern blot), etc. Translation can be determined, for example, by assays of polypeptide expression and/or activity, including immunological assays (e.g., western blot, ELISA), reporter gene transcription assays, reporter protein assays (e.g., translation of a detectable protein such as green fluorescent protein and the like), etc. The level of transcription and/or translation of the X11α and/or X11β nucleic acids in the absence and in the presence of the candidate inhibitor molecule can then be compared. A reduction in the transcription and/or translation of the X11α and/or X11β nucleic acids in the presence of the candidate inhibitor molecule relative to the level of transcription and/or translation in the absence of the candidate inhibitor molecule indicates that the candidate inhibitor molecule is a molecule that reduces production of Aβ. The reaction mixture can be a cell (in vitro or in vivo) or a mixture of isolated and/or purified components.

A typical assay for measuring the rate and/or amount of X11 polypeptide degradation includes one or more X11 polypeptides (i.e., X11α and/or X11β) and protein degradation machinery. Conveniently, assays will use cells that express one or more X11 polypeptides to determine degradation in the context of various components of cellular protein degradation machinery (e.g., proteases, proteasome). Degradation can be determined, for example, by assays of polypeptide expression, stability and/or activity, including immunological assays (e.g., western blot, ELISA), polypeptide binding assays (e.g., between X11 polypeptides and APP), pulse-chase assays, etc. An increase in the rate and/or amount of degradation of the X11 polypeptides in the presence of the candidate degradation enhancer molecule relative to the rate and/or amount of degradation in the absence of the candidate enhancer molecule indicates that the candidate enhancer molecule is a molecule that reduces production of Aβ. The reaction mixture can be a cell (in vitro or in vivo) or a mixture of isolated and/or purified components. Similar assays of nucleic acids can be used to measure the rate and/or amount of X11 nucleic acid degradation.

Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides (e.g., binding sites), and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, polypeptides (including antibodies, antigen-binding fragments of antibodies, fragments of X11 polypeptides, fragments of APP, fusion proteins comprising PTB domain(s) or YENPTY sequence(s) (SEQ ID NO:1)), saccharides, fatty acids, sterols, isoprenoids, purines, pyrimidines, derivatives or structural analogs of the above, or combinations thereof and the like. Where the agent is a nucleic acid (i.e., siRNA, aptamer, gene sequence, protein-encoding sequence, expression vector), the agent typically is a DNA or RNA molecule, although modified nucleic acids having non-natural bonds or subunits are also contemplated.

Candidate agents are obtained from a wide variety of sources including libraries of synthetic or natural compounds. For example, numerous methods are available and known to one of ordinary skill in the art for random and directed synthesis of a wide variety of organic compounds and biomolecules, including expression of randomized oligonucleotides, random or non-random peptide libraries, synthetic organic combinatorial libraries, phage display libraries of random peptides, and the like. Alternatively, libraries of natural compounds in the form of bacterial, fungal, plant and animal extracts are available or readily produced. Libraries of nucleic acids, e.g., polypeptide-encoding sequences, which may be in expression vectors, also are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein activity or stability. Such a reagent may also reduce non-specific or background interactions of the reaction components. Other reagents that improve the efficiency of the assay such as nuclease inhibitors, antimicrobial agents, and the like may also be used.

The mixture of the foregoing assay materials is incubated under conditions whereby, but for the presence of the candidate pharmacological agent, APP binds an X11 polypeptide at a certain level, X11 nucleic acids are transcribed and/or translated at a certain level, or X11 molecules are degraded at a certain rate or amount (i.e., control level). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the presence or absence of binding of the proteins (or binding-competent fragments thereof) the transcription and/or translation of X11 nucleic acids or the degradation of X11 molecules, is detected by any convenient method available to the user. For cell free assays, a separation step may be used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific binding or interaction such as salts, buffer, detergent, non-specific protein, etc. Where the solid substrate is a magnetic bead, the beads may be washed one or more times with a washing solution and isolated using a magnet.

Detection may be effected using any convenient method. For example, one of the components usually comprises, or is coupled to, a detectable label. A wide variety of labels can be used, such as those that provide direct detection (e.g., radioactivity, luminescence, fluorescence, optical or electron density, etc). or indirect detection (e.g., epitope tag such as the FLAG, V5 or myc epitopes, an enzyme tag such as horseradish peroxidase or luciferase, a transcription product, etc.). The label may be bound to a substrate, to the proteins employed in the assays, or to the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the present invention includes automated drug screening assays for identifying molecules having the ability to reduce binding of APP to an X11 polypeptide directly or indirectly, to reduce the transcription and/or translation of X11 nucleic acids or to increase the degradation of X11 molecules. The automated methods preferably are carried out in an apparatus which is capable of delivering a reagent solution to a plurality of predetermined compartments of a vessel and measuring the change in a detectable molecule in the predetermined compartments. Exemplary methods for identifying molecules having the ability to reduce binding of APP to an X11 polypeptide include the following steps. First, a divided vessel is provided that has one or more compartments which contain APP and X11 polypeptides, or binding-competent fragments thereof. The APP and X11 polypeptides can be in a cell in the compartment, in solution, or immobilized within the compartment. Next, one or more predetermined compartments are aligned with a predetermined position (e.g., aligned with a fluid outlet of an automatic pipette) and an aliquot of a solution containing a molecule or mixture of molecules being tested for its ability to reduce APP/X11 binding is delivered to the predetermined compartment(s) with an automatic pipette. Finally, the detectable signal is measured for a predetermined amount of time, preferably by aligning the cell-containing compartment with a detector. Preferably, the signal also is measured prior to adding the compounds to the compartments, to establish e.g., background and/or baseline (control) values. One of ordinary skill in the art can readily determine the appropriate order of addition of the assay components for particular assays. Other similar assays are conducted for identifying molecules having the ability to reduce the transcription and/or translation of X11 nucleic acids or to increase the degradation of X11 molecules.

At a suitable time after addition of the reaction components, the plate is moved, if necessary, so that assay wells are positioned for measurement of signal. Because a change in the signal may begin within the first few seconds after addition of test compounds, it is desirable to align the assay well with the signal detector as quickly as possible, with times of about two seconds or less being desirable. In preferred embodiments of the invention, where the apparatus is configured for detection through the bottom of the well(s) and compounds are added from above the well(s), readings may be taken substantially continuously, since the plate does not need to be moved for addition of reagent. The well and detector device should remain aligned for a predetermined period of time suitable to measure and record the change in signal.

The apparatus of the present invention is programmable to begin the steps of an assay sequence in a predetermined first well (or rows or columns of wells) and proceed sequentially down the columns and across the rows of the plate in a predetermined route through well number n. It is preferred that the data from replicate wells treated with the same compound are collected and recorded (e.g., stored in the memory of a computer) for calculation of signal.

To accomplish rapid addition of molecules and rapid reading of the response, the detector can be modified by fitting an automatic pipetter and utilizing a software program to accomplish precise computer control over both the detector and the automatic pipetter. By integrating the combination of the detector and the automatic pipetter and using a microcomputer to control the commands to the detector and automatic pipetter, the delay time between reagent addition and detector reading can be significantly reduced. Moreover, both greater reproducibility and higher signal-to-noise ratios can be achieved as compared to manual addition of reagent because the computer repeats the process precisely time after time. Moreover, this arrangement permits a plurality of assays to be conducted concurrently without operator intervention. Thus, with automatic delivery of reagent followed by multiple signal measurements, reliability of the assays as well as the number of assays that can be performed per day are advantageously increased.

As used herein a “control” may be a predetermined value, which can take a variety of forms. It can be a single cut-off value, such as a median or mean. It can be established based upon comparative groups, such as groups having a particular disease, condition or symptoms and groups without the disease, condition or symptoms, such as individuals diagnosed with AD and/or elevated production of Aβ and individuals free of AD and/or elevated production of Aβ. Another comparative group would be a group with a family history of a condition and a group without such a family history. The predetermined value can be arranged, for example, where a tested population is divided equally (or unequally) into groups, such as a low-risk group, a medium-risk group and a high-risk group or into quadrants or quintiles.

The predetermined value, of course, will depend upon the particular population selected. For example, an apparently healthy population will have a different ‘normal’ range than will a population which is known to have a condition related to increased Aβ production, APP/X11 binding, and/or transcription and/or translation of X11 nucleic acids, or to decreased degradation of X11 molecules. Accordingly, the predetermined value selected may take into account the category in which an individual falls. Appropriate ranges and categories can be selected with no more than routine experimentation by those of ordinary skill in the art. By abnormal is meant statistically significantly different relative to a selected control. Typically the control will be based on apparently healthy normal individuals in an appropriate age bracket.

In some embodiments, a control sample is from a cell, tissue, or subject that does not have a disorder associated with APP/X11 binding, transcription and/or translation of X11 nucleic acids, degradation of X11 molecules and/or elevated Aβ production. In other embodiments the control sample is a sample that is untreated with a candidate molecule.

The compositions of the present invention may include or be diluted into a pharmaceutically-acceptable carrier. As used herein, “pharmaceutically acceptable carrier” or “physiologically acceptable carrier” means one or more compatible solid or liquid fillers, diluents or encapsulating substances which are suitable for administration to a human or other mammal such as a primate, dog, cat, horse, cow, sheep, or goat. Such carriers include any and all salts, solvents, dispersion media, coatings, antibacterial and antifungal agents, isotonic and absorption delaying agents, and the like that are physiologically compatible. The term “carrier” denotes an organic or inorganic ingredient, natural or synthetic, with which the active ingredient is combined to facilitate the application. The carriers are capable of being co-mingled with the preparations of the present invention, and with each other, in a manner such that there is no interaction which would substantially impair the desired pharmaceutical efficacy or stability. Preferably, the carrier is suitable for oral, intranasal, intravenous, intramuscular, subcutaneous, parenteral, spinal, intradermal or epidermal administration (e.g., by injection or infusion). Suitable carriers can be found in Remington's Pharmaceutical Sciences, Mack Publishing Company, Easton, Pa. Depending on the route of administration, the active molecule, e.g., antibody or siRNA, may be coated in a material to protect the compound from the action of acids and other natural conditions that may inactivate the compound.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. The components of the pharmaceutical compositions also are capable of being co-mingled with the molecules of the present invention, and with each other, in a manner such that there is no interaction which would substantially impair the desired pharmaceutical efficacy. Such preparations may routinely contain salts, buffering agents, preservatives, compatible carriers, and optionally other therapeutic agents, as mentioned elsewhere herein. When used in medicine, the salts should be pharmaceutically acceptable, but non-pharmaceutically acceptable salts may conveniently be used to prepare pharmaceutically-acceptable salts thereof and are not excluded from the scope of the invention.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chioroprocaine, choline, diethanolamine, ethylenediamine, procaine and the like.

The pharmaceutical preparations of the invention also may include isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol.

Optionally, the pharmaceutical preparations of the invention may further comprise a preservative, such as benzalkonium chloride. Suitable preservatives also include but are not limited to: chlorobutanol (0.3-0.9% WNV), parabens (0.01-5.0%), thimerosal (0.004-0.2%), benzyl alcohol (0.5-5%), phenol (0.1-1.0%), and the like.

The formulations provided herein also include those that are sterile. Sterilization processes or techniques as used herein include aseptic techniques such as one or more filtration (0.45 or 0.22 micron filters) steps.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of molecules that reduce binding of APP to X11 polypeptides or reduce the expression of X11 polypeptides (such as siRNA, and/or antibodies, and/or small molecule inhibitors), which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulations suitable for oral, parenteral, subcutaneous, intravenous, intramuscular, etc. administration can be found in Remington's Pharmaceutical Sciences, Mack Publishing Co., Easton, Pa.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The therapeutics of the invention can be administered by any conventional route, including injection or by gradual infusion over time. The administration may, for example, be oral, intravenous, intraperitoneal, intramuscular, intracavity, intracranial, or transdermal. When antibodies are used therapeutically, preferred routes of administration include intravenous and by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

The pharmaceutical preparations of the invention, when used alone or in cocktails, are administered in therapeutically effective amounts. Effective amounts are well known to those of ordinary skill in the art and are described in the literature. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

An “effective amount” is that amount of a molecule that reduces transcription or translation of X11α or X11β, that reduces binding of X11α or X11β to APP, or that increases degradation of X11α or X11β molecules, that alone, or together with further doses, produces the desired response, e.g. reduces production of Aβ and/or treats Alzheimer's disease in a subject. This may involve only slowing the progression of the disease temporarily, although more preferably, it involves halting the progression of the disease permanently. This can be monitored by routine methods. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of anti-X11 molecule antibody, anti-APP antibody, APBA1 or APBA2 siRNA molecule, or other molecule that reduces the expression of X11α a or X11β or the binding of these X11 polypeptides to APP, or that increases degradation of X11α or X11β molecules, for producing the desired response in a unit of weight or volume suitable for administration to a subject. The response can, for example, be measured by determining the physiological effects of the foregoing molecules, such as reduction in Aβ production or decrease of disease symptoms. Other assays will be known to one of ordinary skill in the art and can be employed for measuring the level of the response.

The doses of antibodies, siRNA molecules, or other molecules that reduce expression of X11 molecules or binding of X11 molecules to APP, or that increase degradation of X11α or X11β molecules, administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

Additional treatments for Alzheimer's disease can be used in conjunction with the methods of the invention. Convention AD treatments include, but are not limited to: cholinesterase inhibitors, including donepezil (Aricept®), rivastigmine (Exelon®), galantamine (Reminyl®), and tacrine (Cognex®); NMDA receptor antagonists including memantine (Namenda®); AMPA receptor agonists including CX516 (Ampalex®); choline uptake enhancers, including MKC-231; and HMG CoA reductase inhibitors, i.e., statins. Other treatments include immune therapy.

In addition to being useful for treatment of Alzheimer's disease, the invention also is useful for treating other conditions in which reduced production of Aβ is therapeutically or prophylactically beneficial. Such conditions include: diseases such as Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, and homozygotes for the apolipoprotein E4 allele. Other conditions may be known to those of skill in the art.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, intracranial and infusion.

In general, doses can range from about 10 ng/kg to about 1,000 mg/kg per day, delivered in one or more portions. Based upon the composition, the dose can be delivered continuously, such as by continuous pump, or at periodic intervals. Desired time intervals of multiple doses of a particular composition can be determined without undue experimentation by one skilled in the art. Other protocols for the administration of antibodies, siRNA and other inhibitor molecules will be known to one of ordinary skill in the art, in which the dose amount, schedule of administration, sites of administration, mode of administration and the like vary from the foregoing.

Dosage may be adjusted appropriately to achieve desired drug levels, locally or systemically. Generally, daily oral doses of active compounds will be from about 0.1 mg/kg per day to 30 mg/kg per day. It is expected that i.v. doses in the range of 0.01-1.00 mg/kg will be effective. In the event that the response in a subject is insufficient at such doses, even higher doses (or effective higher doses by a different, more localized delivery route) may be employed to the extent that patient tolerance permits. Continuous i.v. dosing over, for example, 24 hours or multiple doses per day also are contemplated to achieve appropriate systemic levels of compounds.

As used herein, the terms “subject” and “animal” are intended to include humans and non-human animals. Preferred subjects include a human patient who has, is suspected of having, or has a family history of Alzheimer's disease. Other preferred subjects include subjects that are treatable with the compositions of the invention. This includes those who have, are suspected of having, or have a family history of a disease or disorder in which a reduction of Aβ levels is therapeutically or prophylactically beneficial. Administration of the siRNA molecules, antibodies, and other compositions described herein to mammals other than humans, e.g. for testing purposes or veterinary therapeutic purposes, is carried out under substantially the same conditions as described above.

### EXAMPLES

Experimental Procedures

Cell Lines

We employed naïve H4 human neuroglioma (H4) cells and H4 cells stably-transfected to express either the APP-FL, or APP-C99. Peptide APP-C99 is the product of β-secretase, which, therefore, contains α- and γ-, but not β-cleavage sites. This cell line provides a valid system to assess whether any effects on APP processing is dependent on γ-secretase-mediated APP processing and independent of β-secretase-mediated APP processing. All cell lines were cultured in DMEM (high glucose) containing 9% heat-inactivated fetal calf serum, 100 units/ml penicillin, 100 μg/ml streptomycin, and 2 mM L-glutamine. Stably transfected H4 cells were additionally supplemented with 200 μg/ml G418.

RNAi and DAPT Treatment

Small interfering RNA (siRNA) duplexes were designed and obtained from Dharmacon Research, Inc. (Lafayette, CO 80026) against human APBA1, the gene encoding X11α (5′-GGATGCTCAGCTGATTGCA-3′; SEQ ID NO:2), APBA2, the gene encoding X11β (5′-GGTGAAGCTCAACATTGTC-3′; SEQ ID NO:3). Scrambled siRNA (5′-AAATGTGTGTACGTCTCCTCC-3α; SEQ ID NO:4) (29) was used as the control siRNA. siRNAs were transfected into cells by using electroporation (AMAXA, Gaithersburg, Md.). We mixed 1 million cells, 100 μl AMAXA electroporation transfection solution and 10 μl 20 μM siRNA together, then we employed C-9 program in the AMAXA electroporation device for the cell transfection. The transfected cells then were placed in one of the six-well plate containing 1.5 ml cell culture media. The cells were harvested 48 hours after siRNA treatments. DAPT (250 nM, 18 hours treatment time), a γ-secretase inhibitor, was employed in the experiments as a positive control.

Cell Lysis and Protein Amount Quantification

Cell pellets were detergent-extracted on ice using immunoprecipitation buffer (10 mM Tris-HCl, pH 7.4, 150 mM NaCl, 2 mM EDTA, 0.5% Nonidet P-40) plus protease inhibitors (1 μg/ml aprotinin, 1 μg/ml leupeptin, 1 μg/ml pepstatin A). The lysates were collected, centrifuged at 12,000 rpm for 10 min, and quantified for total proteins by the BCA protein assay kit (Pierce, Rockford, Ill.).

Western Blot Analysis of APP Processing

Western blot analysis was performed as described by Xie et al. (30). Briefly, 40 μg of total protein of each sample was subjected to SDS-polyacrylamide gel electrophoresis using 4-20% gradient Tris/glycine gels under reducing conditions (Invitrogen, Carlsbad, Calif.). Next, proteins were transferred to a polyvinylidene difluoride membrane (Bio-Rad, Hercules, Calif.) using a semi-dry electrotransfer system (Amersham Biosciences, San Francisco, Calif.). Nonspecific proteins were blocked using 5% non-fat dry milk in TBST for 1.5 h. Blots were then incubated with a primary antibody, followed by a secondary antibody (horseradish peroxidase-conjugated anti-rabbit antibody 1:10,000; Pierce, Rockford, Ill.). Blots were washed with 1× TBST for 30 min between steps. Antibody H-265 (1:200, Santa Cruz Biotechnology, Santa Cruz, Calif.) was used to recognize X11α (105 kDa), antibody N-20 (1:100, Santa Cruz Biotechnology, Santa Cruz, Calif.) was used to detect X11β (135 kDa). Antibody A8717 (1:1,000, Sigma, St. Louis, Mo.) was used to visualize APP-FL (110 kDa), APP-C83 (12 kDa) and APP-C99 (10 kDa) in the Western blot analysis. The intensity of signals was analyzed using an image program (NIH Image 1.62). We quantified the Western blots as follows. We used the levels of β-actin to normalize the levels of X11α, X11β, APP-FL and APP-CTF (i.e., determining the ratio of X11α amount to β-actin amount) to control for loading differences in total protein amounts. We present the changes in the protein levels of X11α, X11β, APP-FL, APP-C99 and APP-C83 in the cells treated with APBA1 or APBA2 siRNA as the percentage of those in the cells treated with control siRNA.

Quantitation of Aβ Using Sandwich ELISA Assay

Following the treatment with saline, electroporation, control siRNA, APBA1 siRNA or APBA2 siRNA, conditioned media was collected, and secreted Aβ was measured by a Sandwich ELISA assay as described by Xie et al. (30). Briefly, 96-well plates were coated with mouse monoclonal antibodies (mAb) specific to Aβ₄₀ (Ab266) or Aβ₄₂ (21F12). Following blocking with BSA, wells were incubated overnight at 4° C. with test samples of conditioned cell culture media, and then an anti-Aβ (α-Aβ-HR1) conjugated to horseradish peroxidase was added. Plates were then developed with TMB reagent and well absorbance measured at 450 nm. Aβ levels in test samples were determined by comparison with signal from unconditioned media spiked with known quantities of Aβ₄₀ and Aβ₄₂.

Statistics

Analysis of variance (ANOVA) with repeated measurements was employed to compare the difference from the control group. P-values less than 0.05 were considered statistically significant.

Results

APBA1 RNAi Increases APP-CTF Levels and Decreases Aβ Levels in H4-APP-FL Cells

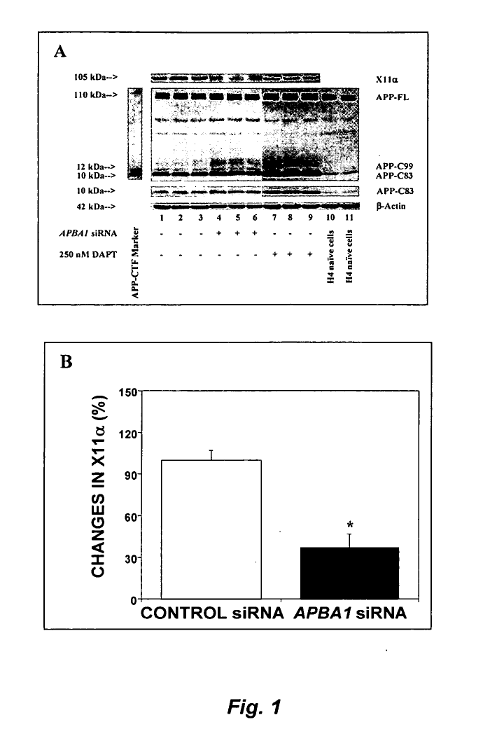

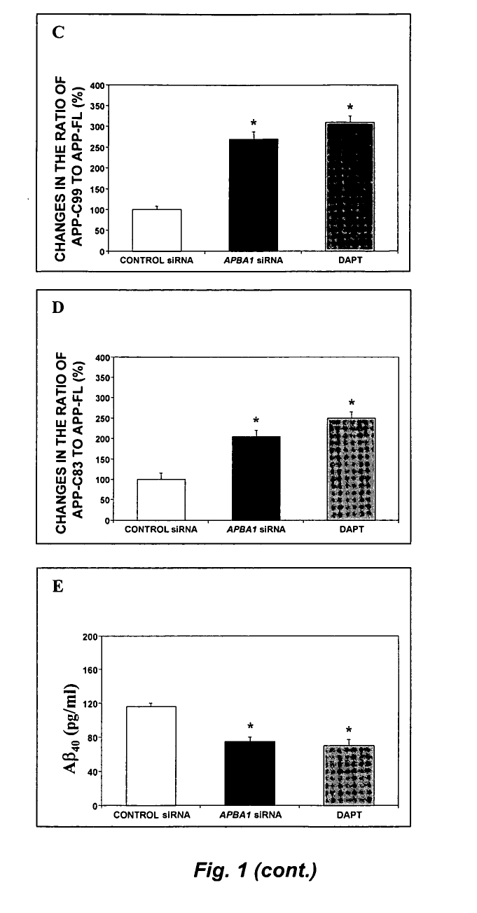

We first established conditions under which APBA1 siRNA treatment would successfully reduce protein levels of X11α in H4 cells over-expressing APP-FL (H4-APP-FL). The cells were harvested 48 hours after being transfected with either control siRNA or APBA1 siRNA, and were subjected to Western blot analyses in which antibody H-265 was used to visualize the protein levels of X11α. As shown in FIG. 1A, X11α immunoblotting revealed visible reduction in the protein levels of X11α following APBA1 siRNA treatment (columns 4 to 6) as compared to control siRNA treatment (columns 1 to 3). There was no significant difference in the amount of β-actin in control siRNA- or APBA1 siRNA-treated cells. We then quantified all the Western blots using the NIH image program. As shown in FIG. 1B, APBA1 siRNA treatment significantly reduced X11α protein levels by 61% (normalized to β-actin) as compared to control siRNA treatment. These results indicated that RNAi for APBA1 significantly knocked down protein levels of X11α.

We next assessed the effects of RNAi-mediated silencing of APBA1 on APP processing in H4-APP-FL cells by measuring protein levels of APP-FL, APP-C99 and APP-C83 following APBA1 siRNA treatment. 48 hours after transfection of APBA1 siRNA or control siRNA, the cells were harvested and subjected to Western blot analyses in which antibody A8717 was used to detect APP-FL, APP-C99 and APP-C83. Protein levels of APP-C99 and APP-C83 were increased in the cells treated with APBA1 siRNA (columns 4 to 6) as compared to control siRNA (columns 1 to 3) (FIG. 1A). As a positive control, the γ-secretase inhibitor DAPT was employed to induce the accumulation of APP-C99 and APP-C83 (columns 7 to 9) as compared to control treatment (columns 1 to 3). In order to confirm bands corresponding to APP-C99 and APP-C83, synthetic forms of APP-C99 and APP-C83 were employed as markers. Meanwhile, no significant differences in the protein levels of APP-FL were observed between APBA1 siRNA-, control siRNA- and DAPT-treated cells. We also assessed protein levels of APP-FL, APP-C99 and APP-C83 in H4 naïve cells, and found the protein levels of APP-FL, APP-C99 and APP-C83 in H4 naïve cells are less than those in H4-APP-FL cells. There was no significant difference in the amount of β-actin in control siRNA-, APBA1 siRNA- or DAPT-treated H4-APP-FL cells, or in H4 naïve cells.

Quantification of APP-FL, APP-C99 and APP-C83 (normalized to β-actin) revealed APBA1 siRNA treatment led to a 270% increase in the ratio of APP-C99 to APP-FL (FIG. 1C, *p<0.05), and a somewhat smaller (205%) increase in the ratio of APP-C83 to APP-FL (FIG. 1D, *p<0.05), as compared to control siRNA treatment. As a control, the γ-secretase inhibitor DAPT led to 310% (FIG. 1C, *p<0.05) and 250% (FIG. 1D, *p<0.05) increases in the ratios of APP-C99 and APP-C83 to APP-FL, respectively.

Next, we measured Aβ levels in the conditioned media, 48 hours after treatment with control siRNA, APBA1 siRNA or γ-secretase inhibitor DAPT. Since Aβ₄₂ was too low to be detected in many samples, we only present the changes in Aβ₄₀ production from these experiments. As can be seen in FIG. 1E, both APBA1 siRNA (black bar) and DAPT (gray bar) decreased Aβ levels to a similar extent as compared to control siRNA treatment (white bar): 75 pg/ml (APBA1 siRNA treatment), 70 pg/ml (DAPT treatment), versus 116 pg/ml (control siRNA treatment). Collectively, these data indicate that RNAi silencing of APBA1 affects APP processing and Aβ production in a manner similar to that of the γ-secretase inhibitor, DAPT.

APBA2 RNAi Decreases Aβ Levels But Does Not Alter APP Processing in H4-APP-FL Cells

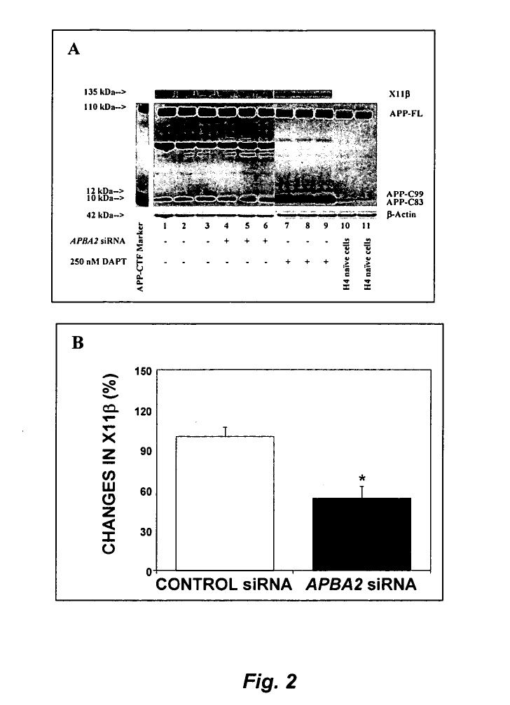

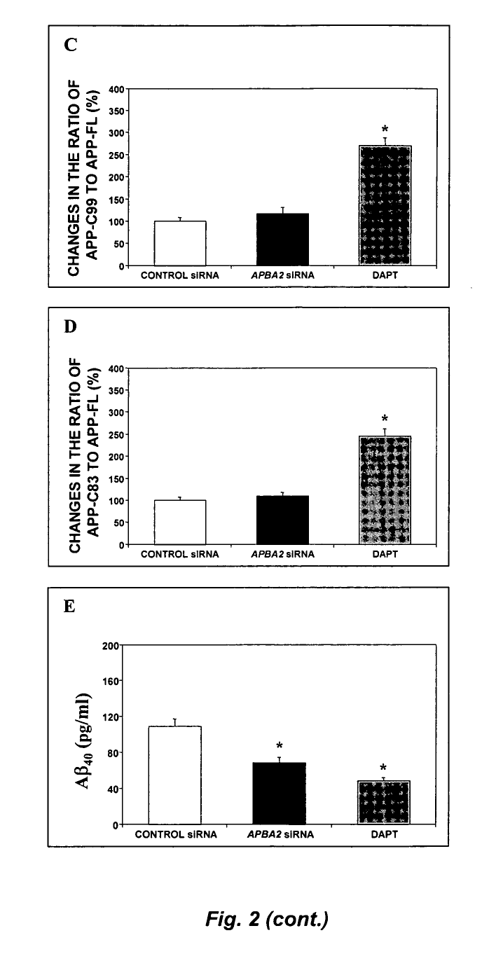

We next asked whether the other X11 family protein, X11β (encoded by gene APBA2) can similarly affect APP processing and Aβ production in H4-APP-FL cells. For this purpose, we established APBA2 RNAi in H4-APP-FL cells. 48 hours after transfection of H4-APP-FL cells with APBA2 siRNA or control siRNA, the cells were harvested and subjected to Western blot analyses in which antibody N-20 was used to detect protein levels of X11β. As shown in FIG. 2A, X11 D immunoblotting revealed a visible decrease in the protein levels of X11β in the cells treated with APBA2 siRNA (columns 4 to 6), as compared to control siRNA (columns 1 to 3). Quantification of the Western blots (normalized to β-actin) revealed that APBA2 siRNA treatment decreased the protein levels of X11β by 46% (FIG. 2B, *p<0.05) as compared to control siRNA treatment.

We then assessed the effects of RNAi-mediated silencing of APBA2 on APP processing in H4-APP-FL cells. 48 hours after transfection with either APBA2 siRNA or control siRNA, the cells were harvested and subjected to Western blot analyses. As shown in FIG. 2A, immunoblotting with antibody A8717 revealed no significant difference in the protein levels of APP-FL in APBA2 siRNA- (columns 4 to 6), control siRNA- (columns 1 to 3) or γ-secretase inhibitor DAPT-treated (columns 7 to 9) cells. In addition, APBA2 siRNA treatment (columns 4 to 6) did not alter protein levels of APP-C99 or APP-C83, whereas γ-secretase inhibitor DAPT treatment (columns 7 to 9), the positive control in the experiment, increased both APP-C99 and APP-C83 levels, as compared to control siRNA treatment (columns 1 to 3) (FIG. 2A). We also assessed protein levels of APP-FL, APP-C99 and APP-C83 in H4 naïve cells, and found the protein levels of APP-FL, APP-C99 and APP-C83 in H4 naïve cells are less than those in H4-APP-FL cells. Quantification of the protein levels of APP-FL, APP-C99 and APP-C83 in the Western blot revealed that γ-secretase inhibitor DAPT treatment (gray bar) led to a 270% (FIG. 2C, *p<0.05) and 245% (FIG. 2D, *p<0.05) increase in the ratio of APP-C99 and APP-C83 to APP-FL, respectively, as compared to control treatment (white bar). However, APBA2 siRNA treatment (black bar) did not alter the ratio of either APP-C99 or APP-C83 relative to APP-FL (FIG. 2D), as compared to control siRNA treatment.

Next, we measured Aβ levels in the conditioned media 48 hours following treatments with control siRNA, APBA2 siRNA or γ-secretase inhibitor DAPT. As can be seen in FIG. 2E, both APBA2 siRNA treatment (black bar) and γ-secretase inhibitor DAPT treatment (gray bar) decreased Aβ production as compared to control siRNA (white bar): 68 pg/ml (APBA2 siRNA), 48 pg/ml (DAPT), 109 pg/ml (control siRNA) (FIG. 2E, *p<0.05). Taken together, these findings indicated that APBA2 siRNA treatment, in contrast to APBA1 siRNA treatment, did not alter APP processing. However, APBA2 siRNA treatment also decreased Aβ production in the H4-APP-FL cells.

As discussed in prior section, β-secretase cleaves APP-FL to produce APP-C99, and then γ-secretase cleaves the APP-C99 to produce Aβ. Therefore, the changes in APP processing and Aβ production following the treatments of APBA1 siRNA and APBA2 siRNA conceivably could be due to alterations in either β-secretase and/or γ-secretase activities. In the following experiments, we set out to determine whether the changes in APP processing and Aβ production following the treatment of APBA1 siRNA or APBA2 siRNA were independent of β-secretase-mediated APP processing and dependent on γ-secretase-mediated APP processing, employing H4 cells over-expressing APP-C99 (H4-APP-C99).

APBA1 RNAi Increases APP-CTF Levels and Decreases Aβ Levels in H4-APP-C99 Cells

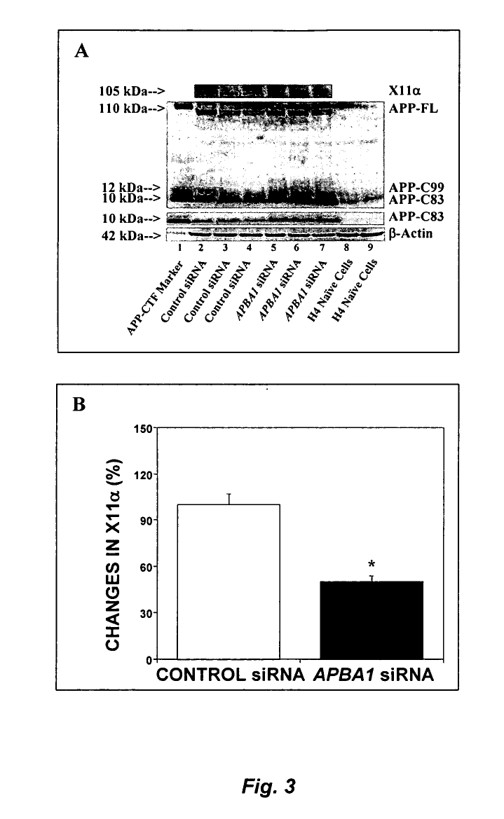

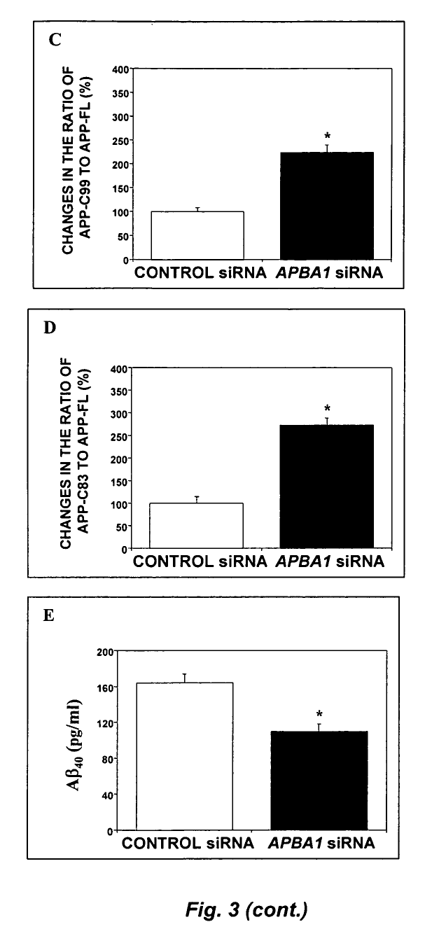

To eliminate increased β-secretase cleavage of APP-FL as a possible explanation for increased APP-C99, we employed H4-APP-C99 cells. APP-C99 is the product of β-secretase and harbors α- and γ-cleavage, but not β-cleavage sites. 48 hours after transfection of H4-APP-C99 cells with APBA1 siRNA or control siRNA, the cells were harvested and subjected to Western blot analyses in which antibody H-265 was used to detect X11α. As shown in FIG. 3A, X11α immunoblotting revealed a visible reduction in the protein levels of X11α, the protein encoded by gene APBA1, in the cells treated with APBA1 siRNA (columns 5 to 7) as compared to control siRNA (columns 2 to 4) (FIG. 3A). There was no significant difference in the amount of β-actin in control siRNA- and APBA1 siRNA-treated cells. Quantification of X11α in the Western blot (normalized to β-actin) showed that APBA1 siRNA treatment decreased the protein levels of X11α by 50% (FIG. 3B, *p<0.05).

We next assessed the effects of RNAi-mediated silencing of APBA1 on processing of APP-C99. 48 hours after transfection with APBA1 siRNA or control siRNA, the cells were harvested and subjected to Western blot analyses with antibody A8717. APBA1 siRNA treatment (columns 5 to 7) did not alter the protein levels of APP-FL as compared to control siRNA treatment (columns 2 to 4) (FIG. 3A). APP-CTF immunoblotting revealed visible increases in the protein levels of both APP-C99 and APP-C83 in the H4-APP-C99 cells treated with APBA1 siRNA (columns 5 to 7), compared to control siRNA (columns 2 to 4) (FIG. 3A). We also assessed protein levels of APP-C99 and APP-C83 in H4 naïve cells, and found the protein levels of APP-C99 and APP-C83 in H4 naïve cells are less than those in H4-APP-C99 cells. There was no significant difference in the amount of β-actin in control siRNA-, or APBA1 siRNA-treated H4 cells over-expressing APP-C99, and in H4 naïve cells. Quantification of APP-FL, APP-C99 and APP-C83 revealed that APBA1 siRNA treatment led to a 224% increase in the ratio of APP-C99 to APP-FL (FIG. 3C, *p<0.05) and a 273% increase in the ratio of APP-C83 to APP-FL (FIG. 3D, *p<0.05), as compared to control siRNA treatment.

We next measured Aβ levels in the conditioned media 48 hours after treatment with either control siRNA or APBA1 siRNA in the H4-APP-C99 cells. As can be seen in FIG. 3E, APBA1 siRNA decreased Aβ production as compared to control siRNA treatment: 110 pg/ml (APBA1 siRNA treatment), 164 pg/ml (control siRNA treatment) (FIG. 3E, *p<0.05). These findings suggest that APBA1 RNAi inhibits γ-secretase-, but not β-secretase-mediated cleavage of APP.

APBA2 RNAi Does Not Alter APP Processing or Aβ Levels in H4-APP-C99 Cells

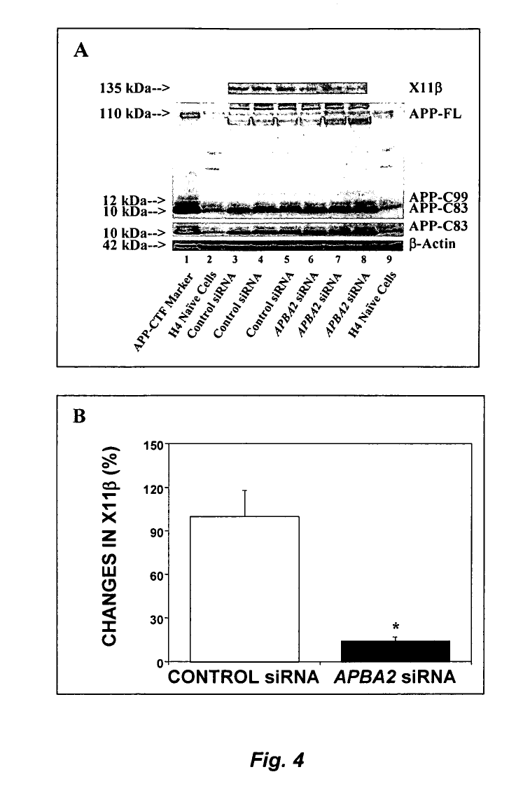

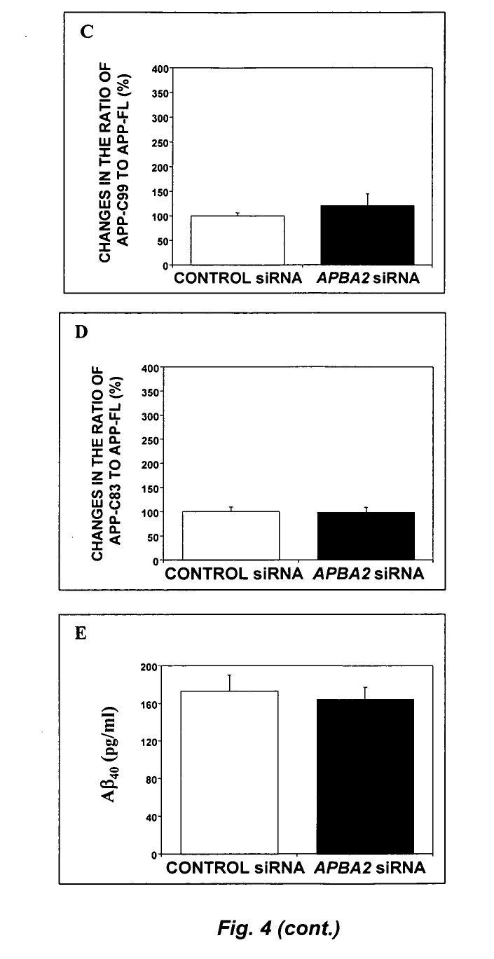

Finally, we assessed the effects of APBA2 RNAi on APP processing and Aβ production in the H4-APP-C99 cells. 48 hours after transfection with APBA2 siRNA or control siRNA, the cells were harvested and subjected to Western blot analyses in which antibody N-20 was used to detect the protein levels of X11β. X11β immunoblotting revealed visible reductions in the protein levels of X11β, the protein encoded by gene APBA2, in the cells treated with APBA2 siRNA (columns 6 to 8) as compared to control siRNA (columns 3 to 5) (FIG. 4A). There was no significant difference in the amount of β-actin in control siRNA- or APBA2 siRNA-treated cells. Quantification of X11β (normalized to β-actin) revealed that APBA2 siRNA treatment decreased the protein levels of X11β by 86% (FIG. 4B, *p<0.05) as compared to control siRNA treatment.

Immunoblotting with APP antibody A8717 revealed no detectable differences in protein levels of APP-FL, APP-C99 or APP-C83 in the cells treated with APBA2 siRNA (columns 6 to 8) as compared to those in the cells treated with control siRNA (columns 3 to 5) (FIG. 4A). We also assessed protein levels of APP-C99 and APP-C83 in H4 naïve cells, and found the protein levels of APP-C99 and APP-C83 in H4 naïve cells are less than those in H4-APP-C99 cells. There was no significant difference in the amount of β-actin in control siRNA-, or APBA2 siRNA-treated H4-APP-C99 cells, and H4 naïve cells. Quantification of the APP-FL, APP-C99 and APP-C83 revealed that APBA2 siRNA treatment did not significantly alter either the ratio of APP-C99 to APP-FL (FIG. 4C) or the ratio of APP-C83 to APP-FL (FIG. 4D). Next, we measured Aβ levels in the conditioned media, 48 hours after treatment with control siRNA or APBA2 siRNA in the H4-APP-C99 cells. As can be seen in FIG. 4E, APBA2 siRNA treatment did not significantly alter Aβ production as compared to control siRNA treatment [(173 pg/ml (control siRNA treatment) versus 167 pg/ml (APBA2 siRNA treatment)] (FIG. 4E). Collectively, these results suggest that in contrast to APBA1 RNAi, APBA2 RNAi did altered neither APP processing nor Aβ production in the H4-APP-C99 cells.

Control siRNA or Electroporation Process Does Not Affect APP Processing or Aβ Levels in H4-APP-FL or H4-APP-C99 Cells.

As a control experiment, we also assessed the effects of control siRNA or electroporation process on APP processing and Aβ levels in H4-APP-FL or H4-APP-C99 cells. We found that control siRNA or electroporation process did not affect APP processing or alter Aβ levels as compared to saline treatment in H4-APP-FL cells or H4-APP-C99 cells (data not shown). These results confirmed that the effects of APBA1 or APBA2 RNAi on APP processing and Aβ levels in our experiments were not due to the electroporation process or control siRNA (scrambled siRNA), but owing to the reduction in the protein levels of X11α and X11β.

Discussion

Aβ, the key component senile plaques, is derived from APP via cleavage by two proteases, β-secretase and γ-secretase (4-7). Cleavage by β-secretase first generates APP-C99, which is further cleaved by γ-secretase to release Aβ and the β-amyloid precursor protein intracellular domain (AICD) (8-10). APP is also cleaved by a-secretase to release a large ectodomain (α-APPs) and APP-C83, APP-C83 is sequentially cleaved by γ-secretase to produce p3 and AICD (11,12), [see review (13)]. Several APP adaptor proteins [see review (21)], including X11α and X11β, have previously been shown to affect APP processing and Aβ production upon over-expression (22-25,27,28,31). Specifically, over-expression in X11α and X11β has been shown to increase APP levels, prolong APP half-life, decrease APPs and decrease Aβ levels (22-25,27,28,31). However, the effects of knock down of X11α and X11β on APP processing and Aβ production have not been previously reported. We have showed for the first time that RNAi silencing of APBA1 and APBA2, the genes encoding X11α and X11β, significantly affects APP processing and Aβ levels.

RNAi silencing of APBA1 led to increased levels of APP-C99 and APP-C83, in the absence of alterations in APP-FL (FIG. 1A, 1C and 1D). Moreover, RNAi for APBA1 significantly decreased Aβ levels (FIG. 1E). Theoretically, the observed increases in APP-C99 and APP-C83 levels following APBA1 siRNA treatment could be due either to increases in the activities of β-secretase and/or α-secretase or to decreases in γ-secretase cleavage of APP-C99 and C83. To discern between these two possibilities, we showed that in H4 cells over-expressing APP-C99, the β-secretase cleavage product of APP, APBA1 siRNA treatment still increased protein levels of APP-C99 and APP-C83 and decreased levels of Aβ in the conditioned media. These data indicate that the observed changes in APP processing and Aβ levels following APBA1 RNAi are independent of β-secretase-, and most likely are due to inhibition of γ-secretase-mediated cleavage of APP.

Next, we asked whether the effects of APBA1 RNAi on γ-secretase-mediated cleavage of APP processing and Aβ production, could be mimicked by RNAi silencing of X11β, encoded by of APBA2. APBA2 RNAi treatment decreased Aβ levels (FIG. 2E), but did not alter the protein levels of APP-FL (FIG. 2A), APP-C99 (FIG. 2A and 2C) or APP-C83 (FIG. 2A and 2D) in the H4-APP-FL cells. In contrast, APBA2 RNAi treatment of H4-APP-C99 cells did not alter the protein levels of APP-FL (FIG. 4A), APP-C99 (FIG. 4A and 4C), APP-C83 (FIG. 4A and 4D), or Aβ levels (FIG. 4E). These findings suggest that unlike the situation with APBA1, the alteration in Aβ levels following RNAi silencing of APBA2 most likely do not involve alterations in γ-secretase cleavage, but may involve changes in β-secretase cleavage of APP. It is also possible that APBA2 RNAi regulates Aβ levels through other mechanisms, such as Aβ degradation. Further studies will be necessary to discern among these possibilities.

Taken together, our findings demonstrate, for the first time, that RNAi knock down of X11α, attenuates γ-secretase-mediated cleavage of APP, leading to increased accumulation of APP-C99 and C83, together with decreased Aβ levels. Previously, over-expression in X11α has also been shown to inhibit APP catabolism (22-25,27,28,31), including increases in levels of APP-C99 and decreases in Aβ levels (28). Thus, it is unexpected that in our current study, RNAi-mediated silencing of X11α, lead to similar, and not opposite effects on APP processing and Aβ levels as compared to the X11α over-expression studies. The phosphotyrosine binding (PTB) domain of X11α [(19), also see review (21)] has been shown to bind the YENPTY motif (SEQ ID NO:1), located in the APP-CTFs, while the PDZ domains of X11α (26) has been reported to bind to presenilin 1 [(14), also see review (21)]. Based on these findings, one might expect that over-expression of X11α may, on one hand, impair clathrin-coated pit internalization of APP by binding the YENPTY motif (SEQ ID NO:1), and consequently lower Aβ levels by blocking entry of APP into the endocytic pathway. And, on the other hand, over-expression of X11α could conceivably enhance γ-secretase cleavage of APP by enhancing its interaction with the γ-secretase component, presenilin 1. Prior studies employing over-expression of X11α reveal the net result to be increased accumulation of APP-CTFs and lower levels of Aβ, consistent with decreased γ-secretase-mediated APP processing. This would suggest that the potential effects of X11α on APP endocytosis outweigh those affecting interactions with presenilin. Further studies will be necessary to investigate this possibility.

Along similar lines, knock down of X11α expression might be expected to attenuate γ-secretase-mediated APP processing based on decreased interaction with presenilin, while potentiating γ-secretase-mediated APP processing by allowing for greater access to the YENPTY motif (SEQ ID NO:1) utilized for clathrin-mediated endocytosis of APP. Our APBA1 RNAi silencing experiments reveal the net result to be similar to that of the previous X11α over-expression studies: increased accumulation of APP-CTF and reduced Aβ levels, owing to decreased γ-secretase-mediated APP processing. Collectively, the combined X11α over-expression and knock down studies suggest that X11α levels must be carefully regulated (neither too high nor too low) to properly maintain normal γ-secretase-mediated APP processing and Aβ production. Interestingly, RNAi silencing of X11β led to a moderate decrease in Aβ levels in the H4-APP-FL cells, but not in the H4-APP-C99 cells. Meanwhile, APBA2 RNAi treatment did not alter APP processing in either cell line, indicating that γ-secretase-mediated APP processing was most likely not affected. One possibility is that the reduction in Aβ levels following APBA2 siRNA treatment involves alterations in either β-secretase-mediated APP processing or Aβ degradation. Further investigation will be necessary to address these possible explanations.

X11α and X11β can also bind to and interact with proteins, other than APP. X11α and X11β can bind to and interact with munc 18-1, a protein essential for synaptic vesicle exocytosis (32). Neurexins are neuron-specific cell surface protein that act as receptors for the excitatory neurotoxin α-lactrotoxin. Both X11α and X11β can bind to the cytoplasmic tail of neurotoxin I, X11α interacts with neurotoxin I via its PDZ domains (32). X11α, but not X11β, also interact with CASK-Veli to form a heterotrimeric complex, mediating transmembrane receptor proteins in polarized cells (33,34). The PDZ domains of X11α may interact with several proteins such as presenilin 1 (26), a presynaptic voltage-gated calcium channel (35), spinophilin-neurabin II (36), the cooper chaperone of SODI (37), the dendritic kinesin KIF-17 (38). Many of these proteins can mediate synaptic function, therefore, suggesting a scaffolding and adaptor function of X11α in the pre- and post-synaptic complex [see review (21)]. As the follow up studies of our initial report, RNAi for X11 affects APP processing and Aβ levels, we will assess the effects of RNAi for X11 on the metabolism of these other X11 interacting proteins in the future. The results of these studies would reveal whether or not attacking X11 could minimize or avoid the likelihood of mechanism-based side effects.

In conclusion, RNAi-mediated silencing of the APP adaptor protein, X11α, attenuates γ-secretase-mediated cleavage of APP leading to decreased Aβ levels. In contrast, RNAi silencing of X11β also lowers Aβ levels, but in the absence of effects on γ-secretase-mediated APP processing. Our findings together with those of previous studies, suggest that pharmaceutical modulation of X11α, X11β, and perhaps other APP adaptor proteins, might potentially serve as a novel therapeutic approach to treating and preventing AD. The notion of attenuating γ-secretase cleavage of APP via APP adaptor proteins, such as X11α, is particularly attractive given that potential unwanted effects of γ-secretase inhibition owing to impaired proteolysis of other γ-secretase substrates, e.g. Notch, might be avoided.

### REFERENCES

1. Tanzi, R. E., and Bertram, L. (2001) Neuron 32, 181-184
2. Selkoe, D. J. (2001) Physiol Rev 81, 741-766
3. Sisodia, S. S., and St George-Hyslop, P. H. (2002) Nat Rev Neurosci 3, 281-290
4. Vassar, R., Bennett, B. D., Babu-Khan, S., Kahn, S., Mendiaz, E. A., Denis, P., Teplow, D. B., Ross, S., Amarante, P., Loeloff, R., Luo, Y., Fisher, S., Fuller, J., Edenson, S., Lile, J., Jarosinski, M. A., Biere, A. L., Curran, E., Burgess, T., Louis, J. C., Collins, F., Treanor, J., Rogers, G., and Citron, M. (1999) Science 286, 735-741
5. Hussain, I., Powell, D., Howlett, D. R., Tew, D. G., Meek, T. D., Chapman, C., Gloger, I. S., Murphy, K. E., Southan, C. D., Ryan, D. M., Smith, T. S., Simmons, D. L., Walsh, F. S., Dingwall, C., and Christie, G. (1999) Mol Cell Neurosci 14, 419-427
6. Sinha, S., and Lieberburg, I. (1999) Proc Natl Acad Sci USA 96, 11049-11053
7. Yan, R., Bienkowski, M. J., Shuck, M. E., Miao, H., Tory, M. C., Pauley, A. M., Brashier, J. R., Stratman, N. C., Mathews, W. R., Buhl, A. E., Carter, D. B., Tomasselli, A. G., Parodi, L. A., Heinrikson, R. L., and Gurney, M. E. (1999) Nature 402, 533-537
8. Gu, Y., Misonou, H., Sato, T., Dohmae, N., Takio, K., and Ihara, Y. (2001) J Biol Chem 276, 35235-35238
9. Sastre, M., Steiner, H., Fuchs, K., Capell, A., Multhaup, G., Condron, M. M., Teplow, D. B., and Haass, C. (2001) EMBO Rep 2, 835-841
10. Yu, C., Kim, S. H., Ikeuchi, T., Xu, H., Gasparini, L., Wang, R., and Sisodia, S. S. (2001) J Biol Chem 276, 43756-43760
11. Esch, F. S., Keim, P. S., Beattie, E. C., Blacher, R. W., Culwell, A. R., Oltersdorf, T., McClure, D., and Ward, P. J. (1990) Science 248, 1122-1124
12. Sisodia, S. S., Koo, E. H., Beyreuther, K., Unterbeck, A., and Price, D. L. (1990) Science 248, 492-495
13. Wolfe, M. S. (2003) Curr Top Dev Biol 54, 233-261
14. Li, Y. M., Lai, M. T., Xu, M., Huang, Q., DiMuzio-Mower, J., Sardana, M. K., Shi, X. P., Yin, K. C., Shafer, J. A., and Gardell, S. J. (2000) Proc Natl Acad Sci USA 97, 6138-6143
15. Francis, R., McGrath, G., Zhang, J., Ruddy, D. A., Sym, M., Apfeld, J., Nicoll, M., Maxwell, M., Hai, B., Ellis, M. C., Parks, A. L., Xu, W., Li, J., Gurney, M., Myers, R. L., Himes, C. S., Hiebsch, R., Ruble, C., Nye, J. S., and Curtis, D. (2002) Dev Cell 3, 85-97
16. Steiner, H., Winkler, E., Edbauer, D., Prokop, S., Basset, G., Yamasaki, A., Kostka, M., and Haass, C. (2002) J Biol Chem 277, 39062-39065
17. Yu, G., Nishimura, M., Arawaka, S., Levitan, D., Zhang, L., Tandon, A., Song, Y. Q., Rogaeva, E., Chen, F., Kawarai, T., Supala, A., Levesque, L., Yu, H., Yang, D. S., Holmes, E., Milman, P., Liang, Y., Zhang, D. M., Xu, D. H., Sato, C., Rogaev, E., Smith, M., Janus, C., Zhang, Y., Aebersold, R., Farrer, L. S., Sorbi, S., Bruni, A., Fraser, P., and St George-Hyslop, P. (2000) Nature 407, 48-54
18. De Strooper, B. (2003) Neuron 38, 9-12
19. Borg, J. P., Ooi, J., Levy, E., and Margolis, B. (1996) Mol Cell Biol 16, 6229-6241
20. McLoughlin, D. M., and Miller, C. C. (1 996) FEBS Lett 397, 197-200
21. King, G. D., and Scott Turner, R. (2004) Exp Neurol 185, 208-219
22. Borg, J. P., Yang, Y., De Taddeo-Borg, M., Margolis, B., and Turner, R. S. (1998) J Biol Chem 273, 14761-14766
23. Sastre, M., Turner, R. S., and Levy, E. (1998) J Biol Chem 273, 22351-22357
24. McLoughlin, D. M., Irving, N. G., Brownlees, J., Brion, J. P., Leroy, K., and Miller, C. C. (1999) Eur J Neurosci 11, 1988-1994
25. Biederer, T., Cao, X., Sudhof, T. C., and Liu, X. (2002) J Neurosci 22, 7340-7351
26. Lau, K. F., McLoughlin, D. M., Standen, C., and Miller, C. C. (2000) Mol Cell Neurosci 16, 557-565
27. King, G. D., Perez, R. G., Steinhilb, M. L., Gaut, J. R., and Turner, R. S. (2003) Neuroscience 120, 143-154
28. King, G. D., Cherian, K., and Turner, R. S. (2004) J Neurochem 88, 971-982
29. Luo, W. J., Wang, H., Li, H., Kim, B. S., Shah, S., Lee, H. J., Thinakaran, G., Kim, T. W., Yu, G., and Xu, H. (2003) J Biol Chem 278, 7850-7854
30. Xie, Z., Moir, R., Romano, D. M., Tesco, G., Kovacs, D., Tanzi. R. (2004) Neurodegenerative Disease 1, 29-37
31. Lee, J. H., Lau, K. F., Perkinton, M. S., Standen, C. L., Shemilt, S. J., Mercken, L., Cooper, J. D., McLoughlin, D. M., and Miller, C. C. (2003) J Biol Chem 278, 47025-47029
32. Biederer, T., and Sudhof, T. C. (2000) J Biol Chem 275, 39803-39806
33. Borg, J. P., Lopez-Figueroa, M. O., de Taddeo-Borg, M., Kroon, D. E., Turner, R. S., Watson, S. J., and Margolis, B. (1999) J Neurosci 19, 1307-1316
34. Whitfield, C. W., Benard, C., Barnes, T., Hekimi, S., and Kim, S. K. (1999) Mol Biol Cell 10, 2087-2100
35. Maximov, A., Sudhof, T. C., and Bezprozvanny, I. (1999) J Biol Chem 274, 24453-24456
36. Ide, N., Hata, Y., Hirao, K., Irie, M., Deguchi, M., Yao, I., Satoh, A., Wada, M., Takahashi, K., Nakanishi, H., and Takai, Y. (1998) Biochem Biophys Res Commun 244, 258-262
37. McLoughlin, D. M., Standen, C. L., Lau, K. F., Ackerley, S., Bartnikas, T. P., Gitlin, J. D., and Miller, C. C. (2001) J Biol Chem 276, 9303-9307
38. Setou, M., Nakagawa, T., Seog, D. H., and Hirokawa, N. (2000) Science 288, 1796-1802

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference.

What is claimed is:

1. A method for reducing the production of Aβ by a cell, comprising
contacting the cell with a molecule that reduces transcription and/or translation of X11α and/or X11β in the cell in an amount effective to reduce the production of Aβ by the cell.

2. The method of claim 1, wherein the molecule is an APBA1 (encoding X11α) and/or APBA2 (encoding X11β) RNAi molecule, optionally wherein the RNAi molecule is a siRNA molecule, and/or optionally wherein the RNAi molecule is formed from two RNA molecules or wherein the RNAi molecule is a single RNA molecule that forms a double stranded (hairpin) structure, or
optionally wherein the molecule is an antisense molecule.

3.-6. (canceled)

7. A method for reducing the production of Aβ by a cell, comprising contacting the cell with a molecule that reduces binding of APP to X11α and/or APP to X11β in the cell.

8. The method of claim 7, wherein the molecule binds to APP and thereby reduces binding of X11α and/or X11β to APP, optionally wherein the molecule is an antibody or an antigen binding fragment thereof that binds to APP, or wherein the molecule is a polypeptide that comprises a PTB domain, or wherein the molecule is a fragment of X11α protein and/or X11β protein that comprises a PTB domain.

9.-11. (canceled)

12. The method of claim 7, wherein the molecule binds to X11α and/or X11β and thereby reduces binding of X11α and/or X11β to APP, optionally wherein the molecule is an antibody or an antigen binding fragment thereof that binds to X11α and/or X11β or wherein the molecule is a polypeptide that comprises a YENPTY sequence (SEQ ID NO:1), preferably wherein the polypeptide is a fragment of APP.

13.-15. (canceled)

16. The method of claim 7, wherein the contacting occurs in vitro.

17. A method for treating or preventing Alzheimer's disease in a subject, comprising
reducing transcription and/or translation of X11α and/or X11β nucleic acids.

18. The method of claim 17, wherein the transcription and/or translation of X11α protein and/or X11β protein is reduced by administering to the subject one or more molecules that bind to X11α and/or X11β nucleic acids and block transcription and/or translation of the X11α and/or X11β nucleic acids, in an amount effective to reduce the transcription and/or translation of X11α and/or X11β nucleic acids and to reduce the production of amyloid β, optionally wherein the one or more molecules that bind to X11α and/or X11β nucleic acids and block transcription and/or translation are APBA1 (encoding X11α) and/or APBA2 (encoding X11β) RNAi molecules,
optionally wherein the RNAi molecule is a siRNA molecule, or wherein the RNAi molecule is formed from two RNA molecules, or wherein the RNAi molecule is a single RNA molecule that forms a double stranded (hairpin) structure, or
optionally wherein the one or more molecules that bind to X11α and/or X11β nucleic acids and block transcription and/or translation are antisense nucleic acid molecules.

19.-23. (canceled)

24. A method for treating or preventing Alzheimer's disease in a subject, comprising
reducing binding of amyloid precursor protein (APP) to X11α protein and/or X11β protein by administering to the subject one or more molecules that bind to APP and block binding of X11α and/or X11β to a YENPTY sequence (SEQ ID NO:1) of APP and/or one or more molecules that bind to X11α and/or X11β and block binding of APP to a phosphotyrosine-binding domain (PTB) of X11α and/or X11β, in an amount effective to reduce the binding of X11α and/or X11β to APP and to reduce the production of amyloid β.

25. The method of claim 24, wherein the molecule is an antibody or an antigen binding fragment thereof that binds to APP protein, optionally wherein the antibody or the antigen binding fragment thereof binds to a YENPTY sequence (SEQ ID NO:1) of APP, or
wherein the molecule is a polypeptide that comprises a YENPTY sequence (SEQ ID NO:1), optionally wherein the molecule is a fragment of APP that comprises a YENPTY sequence (SEQ ID NO:1), or
wherein the molecule is an antibody or an antigen binding fragment thereof that binds to X11α protein and/or X11β protein, optionally wherein the antibody or the antigen binding fragment thereof binds to a PTB domain of X11α protein and/or X11β protein, or
wherein the molecule is a polypeptide that comprises a PTB domain, optionally wherein the molecule is a fragment of X11α protein and/or X11β protein that comprises a PTB domain.

26.-32. (canceled)

33. The method of claim 24, wherein the animal is a human.

34. A method for identifying molecules that reduce production of amyloid β (Aβ) by reducing the binding of amyloid precursor protein (APP) to X11α protein and/or X11β protein, comprising
providing a reaction mixture that comprises APP and/or a fragment thereof that includes a YENPTY sequence (SEQ ID NO:1), and X11α, X11β and/or a fragment thereof that binds to APP,
contacting the reaction mixture with a candidate inhibitor molecule,
determining a level of binding of the APP or the fragment thereof with X11α, X11β and/or the fragment thereof in the absence and in the presence of the candidate inhibitor molecule, and
comparing the level of binding of APP or the fragment thereof with X11α, X11β and/or the fragment thereof in the absence and in the presence of the candidate inhibitor molecule, wherein a reduction in the binding in the presence of the candidate inhibitor molecule relative to the level of binding in the absence of the candidate inhibitor molecule indicates that the candidate inhibitor molecule is a molecule that reduces production of Aβ.

35. The method of claim 34, wherein the candidate inhibitor molecule is a small molecule, or
wherein the candidate inhibitor molecule is an antibody that binds to APP or X11α protein and/or X11β protein, or an antigen-binding fragment thereof, or
wherein the candidate inhibitor molecule is a polypeptide that comprises a YENPTY sequence (SEQ ID NO:1) optionally wherein the candidate inhibitor molecule is a fragment of APP or
wherein the candidate inhibitor molecule is a polypeptide that comprises a phosphotyrosine binding (PTB) domain optionally wherein the candidate inhibitor molecule is a fragment of X11α protein or X11β protein.

36.-40. (canceled)

41. The method of claim 34, wherein the reaction mixture is a cell.

42. A method for identifying molecules that reduce production of amyloid β (Aβ) by reducing transcription and/or translation of X11α and/or X11β, comprising
providing a reaction mixture comprising X11α and/or X11β nucleic acids and transcription and/or translation machinery sufficient to transcribe and/or translate X11α and/or X11β nucleic acids,
contacting the reaction mixture with a candidate inhibitor molecule under conditions that permit transcription and/or translation of the X11α and/or X11β nucleic acids,
determining the transcription and/or translation of the X11α and/or X11β nucleic acids, and
comparing the level of transcription and/or translation of the X11α and/or X11β nucleic acids in the absence and in the presence of the candidate inhibitor molecule, wherein a reduction in the level of transcription and/or translation in the presence of the candidate inhibitor molecule relative to the level of level of transcription and/or translation in the absence of the candidate inhibitor molecule indicates that the candidate inhibitor molecule is a molecule that reduces production of Aβ.

43. The method of claim 42, wherein the candidate inhibitor molecule is a small molecule, or wherein the candidate inhibitor molecule is a siRNA molecule.

44. (canceled)

45. The method of claim 42, wherein the reaction mixture is a cell.

46. A method for identifying molecules that reduce production of amyloid β (Aβ) by increasing degradation of X11 molecules, comprising
providing a cell that expresses X11 molecules,
contacting the cell with a candidate degradation enhancer molecule under conditions that permit degradation of the X11 molecules,
determining the degradation of the X11 molecules, and
comparing the level of degradation of the X11 molecules in the absence and in the presence of the candidate degradation enhancer molecule, wherein an increase of degradation in the presence of the candidate degradation enhancer molecule relative to the level of degradation in the absence of the candidate degradation enhancer molecule indicates that the candidate degradation enhancer molecule is a molecule that reduces production of Aβ.

47. The method of claim 46, wherein the candidate degradation enhancer molecule is a small molecule or
wherein the candidate degradation enhancer molecule is a siRNA molecule, or
wherein the candidate degradation enhancer molecule is a nucleic acid molecule that encodes a polypeptide that increases degradation, optionally wherein the nucleic acid molecule comprises an expression vector, or
wherein the candidate degradation enhancer molecule is a polypeptide that increases degradation.

48.-51. (canceled)

52. The method of claim 46, wherein the X11 molecules are X11α polypeptides and/or X11β polypeptides, or wherein the X11 molecules are X11α nucleic acids and/or X11β nucleic acids.

53. (canceled)


SEQUENCE LISTING (4 sequences)

SEQ ID NO: 1 - protein, 6 aa, Homo sapiens
YENPTY

SEQ ID NO: 2 - DNA, 19 bp, Homo sapiens
ggatgctcagctgattgca

SEQ ID NO: 3 - DNA, 19 bp, Homo sapiens
ggtgaagctcaacattgtc

SEQ ID NO: 4 - DNA, 21 bp, Artificial
  Scrambled siRNA
aaatgtgtgtacgtctcctcc